(19) United States
(12) Patent Application Publication
(10) Pub. No.: US 2014/0295503 A1
(43) Pub. Date: Oct. 2, 2014

(54) METHODS AND DEVICES FOR BIOLOGICAL SAMPLE PREPARATION

(71) Applicant: PathoGenetix, Inc., Woburn, MA (US)

(72) Inventors: Ekaterina Protozanova, Arlington, MA (US); Mohan Nair Manoj Kumar, Burlington, MA (US); Dirk Peter Ten Broeck, Nashua, NH (US); Jimmy Symonds, Nashua, NH (US)

(73) Assignee: PathoGenetix, Inc., Woburn, MA (US)

(21) Appl. No.: 14/208,140

(22) Filed: Mar. 13, 2014

Related U.S. Application Data
(60) Provisional application No. 61/794,560, filed on Mar. 15, 2013.

(51) Int. Cl. C12N 15/10

(52) U.S. Cl. CPC C12N 15/1013
USPC 435/91.53

(57) ABSTRACT

Various aspects and embodiments of the present disclosure relate to methods of obtaining and manipulating nucleic acids from samples. In some embodiments, the samples are known to comprise or are suspected of comprising microorganisms such as bacteria and the methods of the invention are used to identify such microorganisms.

### RELATED APPLICATIONS

This application claims the benefit under 35 U.S.C. §119(e) of U.S. Provisional Application Ser. No. 61/794,560, entitled “METHODS AND DEVICES FOR BIOLOGICAL SAMPLE PREPARATION” filed on Mar. 15, 2013, the entire contents of which are incorporated herein by reference.

### BACKGROUND OF INVENTION

Detecting and optionally identifying and/or quantifying microorganisms in a sample such as, for example, a food sample or a stool sample requires the preparation and concomitant preservation of genomic DNA. Existing techniques for typing microorganisms are often time consuming, laborious and may require operators who have skills in handling DNA samples. Further, these existing techniques are limited by the size of DNA that can be effectively handled. Thus, there exists a need to reduce the time, labor, and/or skills required to prepare essentially intact genomic DNA and other agents of similar length.

### SUMMARY OF INVENTION

The present invention, in its broadest sense, is directed to methods of isolating large, at least partially purified, intact fragments of genomic and plasmid DNA from various samples containing cells such as but not limited to microorganisms including, for example, bacteria, viruses and fungi. The methods of the invention may be used in conjunction with automated reactors such as, for example, those described herein or any of those described in U.S. Pat. No. 8,361,716 B2, U.S. Publication No. 2010/0120101, U.S. Provisional Application No. 61/625,743 (filed Apr. 18, 2012), and Mollova et al. Anal Biochemistry, 391:135-143, 2009, each of which is incorporated by reference herein in its entirety. The resultant isolated DNA fragments are particularly suited for further analysis by a variety of techniques that require high quality, intact DNA.

Thus, various aspects and embodiments of the invention provide methods of isolating nucleic acid using a chamber having a porous substrate, the methods comprising: flowing a sample comprising cells such as microorganisms through a fluid port and onto a porous substrate in the chamber; flowing lytic buffer solution through a fluid port and through the porous substrate in the chamber; flowing a fluid containing lytic reagents through the fluid port and onto the porous substrate in the chamber, and incubating the porous substrate at a first temperature for a first period of time; flowing an endonuclease buffer solution through the fluid port and through the porous substrate in the chamber; flowing a fluid containing digest reagents through the fluid port and onto the porous substrate in the chamber, and incubating the porous substrate at a second temperature for a second period of time; and reversing flow through the fluid port to move any nucleic acids positioned on the porous substrate out of the chamber in central streamlines that exit the chamber through the fluid port, thereby isolating the nucleic acids.

In some embodiments, the lytic reagents comprise a lytic enzyme. In some embodiments, the lytic enzyme is proteinase K, lysostaphin, lysozyme, achromopeptidase, mutanolysin, or any combination of two or more of the foregoing. In some embodiments, the lytic reagents further comprise a buffer, a denaturing agent, a detergent, a chelating agent, a reducing agent, or any combination of two or more of the foregoing.

In some embodiments, the first temperature is about 37° C. to about 75° C. In some embodiments, the first period of time is about 5 minutes to about 30 minutes, including about 10 minutes to about 30 minutes.

In some embodiments, the lytic step is performed multiple times. In some embodiments, multiple different lytic enzymes are used and multiple different lytic buffer solutions are used.

In some embodiments, the methods further comprise flowing a fluid containing lytic reagents through the fluid port and onto the porous substrate in the chamber, wherein the lytic reagents comprise a first lytic enzyme and a first lytic buffer solution, and incubating the porous substrate; and flowing a fluid containing lytic reagents through the fluid port and onto the porous substrate in the chamber, wherein the lytic reagents comprise a second lytic enzyme different from the first lytic enzyme and a second lytic buffer solution different from the first lytic buffer solution, and incubating the porous substrate.

In some embodiments, the digest reagents comprise an endonuclease. In some embodiments, the endonuclease is PmeI, XbaI, ApaI, or any combination of two or more of the foregoing.

In some embodiments, the digest reagents further comprise magnesium, sodium, potassium, salt, tris(hydroxymethyl)aminomethane, or any combination of two or more of the foregoing.

In some embodiments, the second temperature is about 20° C. to about 37° C. In some embodiments, the second period of time is about 5 minutes to about 30 minutes, including about 10 minutes to about 30 minutes.

In some embodiments, the digest step is performed multiple times. In some embodiments, multiple different endonucleases are used and multiple different endonuclease buffer solutions are used.

In some embodiments, the methods further comprise flowing a fluid containing digest reagents through the fluid port and onto the porous substrate in the chamber, wherein the digest reagents comprise a first endonuclease and a first endonuclease buffer solution, and incubating the porous substrate; and flowing a fluid containing digest reagents through the fluid port and onto the porous substrate in the chamber, wherein the digest reagents comprise a second endonuclease different from the first endonuclease and a second endonuclease buffer solution different from the first endonuclease buffer solution, and incubating the porous substrate.

In some embodiments, the methods further comprise flowing low salt wash buffer through the fluid port and through the porous substrate in the chamber, flowing a fluid containing nucleic acid probe through the fluid port and onto the porous substrate in the chamber, incubating the porous substrate at a third temperature for a third period of time, flowing high salt wash buffer through the fluid port and on the porous substrate in the chamber, incubating the porous substrate at a fourth temperature for a fourth period of time, and flowing low salt wash buffer through the fluid port and through the porous substrate in the chamber.

In some embodiments, the sample is pre-treated to remove matrix. In some embodiments, the matrix is removed by sedimentation, selective sedimentation, density gradient centrifugation or filtration.

In some embodiments, the sample is a biological sample. In some embodiments, the sample is food sample or a stool sample.

In some embodiments, the nucleic acids have a length of at least 50 kilobases (kb). In some embodiments, the nucleic acids have a length of at least 100 kilobases, at least 150 kilobases, at least 250 kilobases, at least 500 kilobases, at least 750 kilobases, at least 1 megabase, or at least 5 megabases.

In some embodiments, the nucleic acids are isolated in 12 hours or less. In some embodiments, the nucleic acids are isolated in 6 hours or less, 5 hours or less, 4 hours or less, or 3 hours or less.

In some embodiments, the microorganisms are bacteria, fungi, virus or a combination of any two or more of the foregoing.

In some embodiments, the nucleic acids are not in vitro amplified.

In some embodiments, the sample is not cultured prior to flowing the sample through the fluid port.

In some embodiments, the porous substrate is a membrane. In some embodiments, the membrane is an ultrafiltration membrane.

In various other aspects and embodiments of the invention, provided herein are methods of isolating nucleic acid using a chamber having a porous substrate, the method comprising: flowing a sample comprising cells such as microorganisms through a fluid port and onto a porous substrate in the chamber; flowing lytic buffer solution through a fluid port and through the porous substrate in the chamber; flowing a fluid containing lytic reagents through the fluid port and onto the porous substrate in the chamber, and incubating the porous substrate at a first temperature for a first period of time; flowing a first endonuclease buffer solution through the fluid port; flowing a fluid containing digest reagents through the fluid port off-center and onto the first half of the porous substrate in the chamber, and incubating the porous substrate at a second temperature for a second period of time; reversing flow through the fluid port to move any nucleic acids positioned on the first half of porous substrate out of the chamber through the fluid port, thereby isolating digested nucleic acids on the first half of the porous substrate; flowing a second endonuclease buffer solution through the fluid port; flowing a fluid containing digest reagents through the fluid port off-center and onto the second half of the porous substrate in the chamber, and incubating the porous substrate at a third temperature for a third period of time; and reversing flow through the fluid port to move any nucleic acids positioned on the second half of porous substrate and therefore digested with second digest reagent out of the chamber through the fluid port, thereby isolating the nucleic acids from the second half of the porous substrate.

In some embodiments, the lytic reagents comprise a lytic enzyme. In some embodiments, the lytic enzyme is proteinase K, lysostaphin, lysozyme, achromopeptidase, mutanolysin, or any combination of two or more of the foregoing. In some embodiments, the lytic reagents further comprise a denaturing agent, a detergent, a chelating agent, a reducing agent, or any combination of two or more of the foregoing.

In some embodiments, the first temperature is about 37° C. to about 75° C. In some embodiments, the first period of time is about 5 minutes to about 30 minutes, including about 10 minutes to about 30 minutes.

In some embodiments, the lytic step is performed multiple times. In some embodiments, multiple different lytic enzymes are used and multiple different lytic buffer solutions are used.

In some embodiments, the methods further comprise flowing a fluid containing lytic reagents through the fluid port and onto the porous substrate in the chamber, wherein the lytic reagents comprise a first lytic enzyme and a first lytic buffer solution, and incubating the porous substrate; and flowing a fluid containing lytic reagents through the fluid port and onto the porous substrate in the chamber, wherein the lytic reagents comprise a second lytic enzyme different from the first lytic enzyme and a second lytic buffer solution different from the first lytic buffer solution, and incubating the porous substrate.

In some embodiments, the digest reagents comprise an endonuclease. In some embodiments, the endonuclease is PmeI, XbaI, ApaI, or any combination of two or more of the foregoing.

In some embodiments, the digest reagents further comprise magnesium, sodium, potassium, salt, tris(hydroxymethyl)aminomethane, or any combination of two or more of the foregoing.

In some embodiments, the second temperature is about 20° C. to about 37° C. In some embodiments, the second period of time is about 5 minutes to about 30 minutes, including about 10 minutes to about 30 minutes.

In some embodiments, the digest reagents of one digest step comprise an endonuclease and an endonuclease buffer solution different from those in another digest step.

In some embodiments, the third temperature is about room temperature to about 37° C. In some embodiments, the third period of time is about 5 minutes to about 30 minutes, including about 10 minutes to about 30 minutes.

In some embodiments, the sample is pre-treated to remove matrix. In some embodiments, the matrix is removed by sedimentation, selective sedimentation, density gradient centrifugation or filtration.

In some embodiments, the sample is a biological sample. In some embodiments, the sample is food sample or a stool sample.

In some embodiments, the nucleic acids have a length of at least 50 kilobases (kb). In some embodiments, the nucleic acids have a length of at least 100 kilobases, at least 150 kilobases, at least 250 kilobases, at least 500 kilobases, at least 750 kilobases, at least 1 megabase, or at least 5 megabases.

In some embodiments, the nucleic acids are isolated in 12 hours or less. In some embodiments, the nucleic acids are isolated in 6 hours or less, 5 hours or less, 4 hours or less, or 3 hours or less.

In some embodiments, the microorganisms are bacteria, fungi, virus or a combination of any two or more of the foregoing.

In some embodiments, the nucleic acids are not in vitro amplified.

In some embodiments, the sample is not cultured prior to flowing the sample through the fluid port.

In some embodiments, the porous substrate is a membrane. In some embodiments, the membrane is an ultrafiltration membrane.

In yet other aspects and embodiments of the invention, provided herein are methods of conjugating nucleic acids using a chamber having a porous substrate, the method comprising: flowing a fluid containing at least two populations of nucleic acids through a fluid port and onto a porous substrate in the chamber, wherein the length of the nucleic acids in one population is at least 10-fold longer than the length of the nucleic acids in the other population; flowing a fluid containing a nucleic acid ligase and ligase buffer through the fluid port and onto the porous substrate in the chamber; incubating the porous substrate for a period of time and temperature that permit nucleic acid ligation; and reversing flow through the fluid port to move any nucleic acids positioned on the porous substrate out of the chamber in central streamlines that exit the chamber through the fluid port, thereby isolating conjugated nucleic acids.

In some embodiments, the length of the nucleic acids in one population is at least 100-fold or at least 1000-fold longer than the length of the nucleic acids in the other population.

In still other aspects and embodiments of the invention, provided herein are compositions comprising a plurality of nucleic acids, wherein at least about 50% of the nucleic acids have lengths in the range of about 100 kb to about 1000 kb.

In some embodiments, at least about 60%, at least about 70%, at least about 80%, at least about 90%, or at least about 100% of the nucleic acids have lengths in the range of about 100 kb to about 1000 kb.

In some aspects and embodiments of the invention, provided herein are methods of isolating nucleic acid using a chamber having a porous substrate, the method comprising: flowing a sample comprising cells such as microorganisms through a fluid port and onto a porous substrate in the chamber; flowing lytic buffer solution through a fluid port and through the porous substrate in the chamber; flowing a fluid containing lytic reagents through the fluid port and onto the porous substrate in the chamber, and incubating the porous substrate at a set temperature for a set period of time; incubating the porous substrate at a temperature of about 65° C. to about 75° C. for a time sufficient to permit melting of AT-rich regions of the nucleic acid; and reversing flow through the fluid port to move any nucleic acids positioned on the porous substrate out of the chamber in central streamlines that exit the chamber through the fluid port, thereby isolating the nucleic acids.

In some embodiments, a portion of the population contains nucleic acid fragments having lengths in the range of about 100 kb to about 1000 kb.

In some embodiments, the portion that contains nucleic acid fragments having lengths in the range of about 100 kb to about 1000 kb represents at least about 50%, at least about 60%, at least about 70%, at least about 80%, at least about 90%, or at least about 100% of the population.

In some embodiments, the porous substrate is incubated for a period of time of about 5 minutes to about 30 minutes, including about 10 minutes to about 30 minutes.

In other aspects and embodiments of the invention, provided herein are methods of isolating nucleic acid using a chamber having a porous substrate, the method comprising: flowing a sample comprising cells such as microorganisms through a fluid port and onto a porous substrate in the chamber; flowing lytic buffer solution through a fluid port and through the porous substrate in the chamber; flowing a fluid containing lytic reagents through the fluid port and onto the porous substrate in the chamber, and incubating the porous substrate at a set temperature for a set period of time; aspirating solution from the chamber and depositing the solution onto the porous substrate, and repeating the aspirating and depositing multiple times thereby shearing the nucleic acid; and reversing flow through the fluid port to move any nucleic acids positioned on the porous substrate out of the chamber in central streamlines that exit the chamber through the fluid port, thereby isolating the nucleic acids.

In some embodiments, a portion of the population contains nucleic acid fragments having lengths in the range of about 100 kb to about 1000 kb.

In some embodiments, the portion that contains nucleic acid fragments having lengths in the range of about 100 kb to about 1000 kb represents at least about 50%, at least about 60%, at least about 70%, at least about 80%, at least about 90%, or at least about 100% of the population.

In some embodiments, the porous substrate is incubated for a period of time of about 5 minutes to about 30 minutes, including about 10 minutes to about 30 minutes.

In other aspects and embodiments of the invention, provided herein are methods of producing fragmented nucleic acids using a chamber having a porous substrate, the method comprising: disposing high molecular weight nucleic acid onto a porous substrate in the chamber; incubating the porous substrate at a temperature of about 65° C. to about 75° C. for a time sufficient to permit melting of AT-rich regions of the nucleic acid; and reversing flow through the fluid port to move any nucleic acids positioned on the porous substrate out of the chamber in central streamlines that exit the chamber through the fluid port, thereby isolating a population of nucleic acid fragments.

In some embodiments, a portion of the population contains nucleic acid fragments having lengths in the range of about 100 kb to about 1000 kb. In some embodiments, the portion that contains nucleic acid fragments having lengths in the range of about 100 kb to about 1000 kb represents at least about 50%, at least about 60%, at least about 70%, at least about 80%, at least about 90%, or at least about 100% of the population.

In some embodiments, the porous substrate is incubated for a period of time of about 5 minutes to about 30 minutes, or about 10 minutes to about 30 minutes.

Various embodiments of the invention may be performed using a reactor such as an automated reactor. The reactors may include a body having a chamber with an inlet and a porous substrate (e.g., membrane) positioned in the body. The porous substrate may have a first side and a second side, where the inlet is positioned on the first side of the porous substrate. The reactors may also include a plurality of channels coupled to the bottom of the chamber, where the plurality of channels are positioned on the second side of the porous substrate. Each of the plurality of channels may extend outwardly from the porous substrate, the plurality of channels including at least a first channel and a second channel, where the first channel may extend outwardly from a central portion of the porous substrate, and where the second channel may extend outwardly from a peripheral portion of the porous substrate.

In some embodiments, the reactors may include a body having a chamber with an inlet and a porous substrate positioned in the body. The porous substrate may have a first side and a second side, where the inlet is positioned on the first side of the porous substrate. The porous substrate may include at least a first zone and a second zone, where the first zone is the central portion of the porous substrate and the second zone is the peripheral portion of the porous substrate and there is a barrier which separates the first zone of the porous substrate from the second zone of the porous substrate. The reactors may also include a plurality of channels coupled to the bottom of the chamber, where the plurality of channels are positioned on the second side of the porous substrate.

### BRIEF DESCRIPTION OF DRAWINGS

FIG. 1 shows fluorescent traces generated from Escherichia coli (E. coli) K12 DNA digested with SanDI restriction enzyme and fluorescent probes complementary to 5′-AGAAAGAG (top 2 traces of each four trace set) and AAGAGAAG (bottom 2 traces of each four trace set). Positions of these fragment on the E. coli K12 genome and their lengths are also indicated. For each fragment, a 4-trace set is provided. Each 4-trace set comprises two 2-trace sets (with the top and bottom 2-trace sets described above). Within each 2-trace set, the top trace is an experimental trace and the bottom trace is a theoretical trace.

FIG. 2 shows a schematic of the major functions of an automated reactor utilized to produce high quality DNA.

FIGS. 3A-3B show photographs depicting a pretreatment protocol of the invention, in which a ground beef sample is cultured overnight. FIG. 3A shows sedimentation of particulate matter due to density difference on bench top. FIG. 3B shows that slow centrifugation further cleans the sample by removing debris from the matrix. FIG. 3C shows clean bacterial cells that are pelleted by fast centrifugation. FIG. 3D shows bacterial cells re-suspended in solution for, as an example, subsequent injection into a reactor.

FIG. 4 shows photographs of a fecal sample before and after centrifugation through a HistoDenz™ density gradient of 10-15-30. The rectangle indicates the 15-30% interface from which bacterial cells are collected.

FIG. 5 shows photographs of mold hyphae separated by density gradient centrifugation.

FIGS. 6A-6C show an example of a protocol used to obtain two restriction digests of DNA using the same reactor. A probe is position off-center, to the left, in the reactor (FIG. 6A) to deposit a first restriction digest, resulting in only partial coverage of DNA on the membrane (FIG. 6B, left). The probe is then position off-center, to the right, in the reactor to deposit a second restriction digest, resulting in partial coverage of DNA on the membrane (FIG. 6B, right). FIG. 6C shows an agarose gel of a PFGE pattern of E. coli K12 DNA digested with PmeI and XbaI in one reactor (as depicted in FIGS. 6A and 6B), showing different band patterns expected for corresponding digests.

FIGS. 7A-7C show electrophoresis gels with DNA elution profiles from a reactor. DNA was eluted from the reactor mostly in one fraction following digestion by restriction enzyme. (FIG. 7A, lane 3). In the absence of restriction enzyme digestion, DNA did not elute (FIG. 7B). In later fractions, after high temperature treatment, DNA was efficiently eluted from the reactor (FIG. 7C, lanes 4, 5 and 6).

FIGS. 8A-8C show Genome Sequence Scanning™ (GSS™) fluorescent traces of 200 kb fragments of Micrococcus leteus (73% GC) that were obtained with fluorescent probes complementary to 5′-GAAGAAAA (FIG. 8A), 5′-GAAGAAGG (FIG. 8B) and a mixture of 5′-GAAGAAAA and 5′-GAAGAAGG (FIG. 8C).

FIG. 9 shows GSS fluorescent traces for long fragments of genomic DNA obtained using one set of sequence-specific probes designed to target genomes with a very wide range of GC contents. Tags labeled with ATTO550 dye, which are complementary to 5′-GAAGAAAA and 5′-GAAGAAGG, act together with probes labeled with ATTO647N dye, which are complementary to 5′-GAAAAAGA and 5′-AGAAGAAG probes.

FIG. 10 shows a schematic representation of the major steps of an automated sample preparation protocol. DNA prepared this way may be eluted at the steps indicated for further analysis.

FIG. 11 shows the length distribution of DNA eluted from a reactor, without a restriction enzyme digestion step, following pipetting and/or incubation at high temperature.

FIG. 12 shows a gel with pulse field gel electrophoresis (PFGE) band patterns of high molecular weight DNA extracted from various bacteria using a sample preparation protocol of the invention in an automated reactor.

FIG. 13 shows an electrophoresis gel of a PFGE band pattern of high molecular weight bacterial DNA isolated from ground beef sample. Ground beef was cultured according to USDA protocol following by a sample preparation protocol of the invention prior to injection in an automated reactor. Lane 2 shows PFGE band pattern of DNA obtained from a sample of ground beef spiked with 10³ cfu of Salmonella. Lane 1 shows PFGE band pattern of DNA obtained from a sample of ground beef, without bacterial spiking.

FIG. 14 shows a GSS analysis of bacteria, yeast and mold. The heat map on the left shows the distribution of high molecular weight DNA, similar to PFGE. The plot on the right shows sequence-specific signal from bisPNA tags bound to genomic DNA.

### DETAILED DESCRIPTION OF INVENTION

The present invention provides, inter alia, methods of obtaining large (e.g., >50 kilobases), at least partially purified, intact fragments of genomic and plasmid DNA from biological samples (e.g., food samples or stool samples) comprising cells including for example microorganisms such as, for example, bacteria, viruses and/or fungi. For the purposes of this invention, viruses, fungi, and bacteria are all considered cells. The microorganisms may be, for example, pure samples/cultures (consisting of one microorganism) or complex samples/cultures (comprising more than one microorganism) and may be processed without a priori knowledge of the sample/culture composition. The methods of the invention may be used in conjunction with automated reactors such as, but not limited to, any of those described in U.S. Pat. No. 8,361,716 B2, U.S. Publication No. 2010/0120101, U.S. Provisional Application No. 61/625,743 (filed Apr. 18, 2012), and Mollova et al. Anal Biochemistry, 391:135-143, 2009, each of which is incorporated by reference herein in its entirety. It is to be understood that the invention contemplates the use of other reactor systems including other automated reactor systems, and is not limited in this regard.

The resultant isolated DNA fragments are particularly suited for further analysis by a variety of techniques that require high quality, intact DNA including, without limitation, polymerase chain reaction (PCR) (or other techniques where purified DNA is analyzed), pulse-field gel electrophoresis (PFGE) (or other techniques where long, intact purified DNA is analyzed), Genome Sequence Scanning™ (GSS™) (or other techniques where purified, long, intact sequence-specific labeled DNA is analyzed).

### Biological Samples

The biological samples analyzed in accordance with the invention include, without limitation, biological specimen, clinical specimen and food samples. In some embodiments, the sample is a cultured isolate. In some embodiments, the sample is not cultured. The volume of sample that is injected into an automated reactor may be, in some embodiments, about 0.1 ml to about 5 ml.

In some embodiments, the sample is a suspension of microorganisms in water, buffer or broth. Suspensions may be prepared by resuspending microorganisms from one or more colonies of an agar plate, by resuspending a pellet after centrifugation, or may be used directly following culturing microorganism in media and ambient conditions specific to the application. Sample types described above typically do not contain matrices and do not require pre-treatment, as provided herein (e.g., prior to deposition in an automated reactor).

Examples of biological specimen include, without limitation, fungi (e.g., mold) and yeast, which when cultured, may require separation of lysis-resistant hyphae from the culture.

Examples of clinical specimen include, without limitation, stool samples (diurrheal and/or normal), urine samples, tissue/cell samples (e.g., biopsies), and blood samples.

Examples of food samples (e.g., FDA, food category III) include, without limitation, whole grain, milled grain products that are cooked before consumption (corn meal and all types of flour), and starch products for human use, prepared dry mixes for cakes, cookies, breads, and rolls, macaroni and noodle products, fresh and frozen fish; vertebrates; fresh and frozen shellfish and crustaceans; other aquatic animals (including frog legs, marine snails, and squid), vegetable protein products (simulated meats) normally cooked before consumption, fresh vegetables, frozen vegetables, dried vegetables, cured and processed vegetable products normally cooked before consumption, vegetable oils, oil stock, and vegetable shortening, dry dessert mixes, pudding mixes, and rennet products that are cooked before consumption.

Other examples of food samples (e.g., FDA, food categories I and II) include, without limitation, milled grain products not cooked before consumption (bran and wheat germ); bread, rolls, buns, sugared breads, crackers, custard- and cream-filled sweet goods, and icings, breakfast cereals and other ready-to-eat breakfast foods; pretzels, chips, and other snack foods; butter and butter products; pasteurized milk and raw fluid milk and fluid milk products for direct consumption; pasteurized and unpasteurized concentrated liquid milk products for direct consumption; dried milk and dried milk products for direct consumption, casein, sodium caseinate, and whey; cheese and cheese products; ice cream from pasteurized milk and related products that have been pasteurized, raw ice cream mix and related unpasteurized products for direct consumption; pasteurized and unpasteurized imitation dairy products for direct consumption; pasteurized eggs and egg products from pasteurized eggs, unpasteurized eggs and egg products from unpasteurized eggs for consumption without further cooking; canned and cured fish, vertebrates, and other fish products; fresh and frozen raw shellfish and crustacean products for direct consumption; smoked fish, shellfish, and crustaceans for direct consumption; meat and meat products, poultry and poultry products, and gelatin (flavored and unflavored bulk); fresh, frozen, and canned fruits and juices, concentrates, and nectars; dried fruits for direct consumption; jams, jellies, preserves, and butters; nuts, nut products, edible seeds, and edible seed products for direct consumption; vegetable juices, vegetable sprouts, and vegetables normally eaten raw; oils consumed directly without further processing; oleomargarine; dressings and condiments (including mayonnaise), salad dressing, and vinegar; spices, flavors, and extracts; soft drinks and water; beverage bases; coffee and tea; candy (with and without chocolate; with and without nuts) and chewing gum; chocolate and cocoa products; pudding mixes not cooked before consumption, and gelatin products; syrups, sugars, and honey; ready-to-eat sandwiches, stews, gravies, and sauces; soups; prepared salads; and nutrient supplements, such as vitamins, minerals, proteins and dried inactive yeast.

Additional examples of food samples for use in accordance with the invention may be found in United States Department of Agriculture, Food Safety and Inspection Service, Office of Public Health Science, Laboratory Guidebook Notice of Change, effective Oct. 8, 2010, at the FSIS/USDA website); and in the Bacteriological Analytical Manual (BAM) found on the U.S. Food and Drug Administration website.

### Microorganisms

The methods provided herein may be used to detect and optionally identify and/or quantify any microorganism, including rare microorganisms that would be costly to detect given the reagents necessary therefor. One example of such microorganisms are pathogens (including spores thereof). As used herein, a pathogen (including a spore thereof) is an microorganism capable of entering a subject such as a human and infecting that subject. Examples of pathogens include infectious agents such as bacteria, viruses, fungi, parasites, mycobacteria and the like. Examples of pathogens are provided below.

CDC Category A pathogens include Bacillus anthracis (otherwise known as anthrax), Clostridium botulinum and its toxin (causative agent for botulism), Yersinia pestis (causative agent for the plague), variola major (causative agent for small pox), Francisella tularensis (causative agent for tularemia), and viral hemorrhagic fever causing agents such as filoviruses Ebola and Marburg and arenaviruses such as Lassa, Machupo and Junin.

CDC Category B pathogens include Brucellosis (Brucella species), epsilon toxin of Clostridium perfringens, food safety threats such as Salmonella species, E. coli and Shigella, Glanders (Burkholderia mallei), Melioidosis (Burkholderia pseudomallei), Psittacosis (Chlamydia psittaci), Q fever (Coxiella burnetii), ricin toxin (from Ricinus communis—castor beans), Staphylococcal enterotoxin B, Typhus fever (Rickettsia prowazekii), viral encephalitis (alphaviruses, e.g., Venezuelan equine encephalitis, eastern equine encephalitis and western equine encephalitis), and water safety threats such as e.g., Vibrio cholera and Cryptosporidium parvum.

CDC Category C pathogens include emerging infectious diseases such as Nipah virus and hantavirus.

Additional examples of bacteria that may be harvested and/or manipulated according to the invention include Gonorrhea, Staphylococcus spp., Streptococcus spp. such as Streptococcus pneumoniae, Syphilis, Pseudomonas spp., Clostridium difficile, Legionella spp., Pneumococcus spp., Haemophilus spp. (e.g., Haemophilus influenzae), Klebsiella spp., Enterobacter spp., Citrobacter spp., Neisseria spp. (e.g., N. meningitidis, N. gonorrhoeae), Shigella spp., Salmonella spp., Listeria spp. (e.g., L. monocytogenes), Pasteurella spp. (e.g., Pasteurella multocida), Streptobacillus spp., Spirillum spp., Treponema spp. (e.g., Treponema pallidum), Actinomyces spp. (e.g., Actinomyces israelli), Borrelia spp., Corynebacterium spp., Nocardia spp., Gardnerella spp. (e.g., Gardnerella vaginalis), Campylobacter spp., Spirochaeta spp., Proteus spp. and Bacteroides spp.

Additional examples of viruses that may be harvested and/or manipulated according to the invention include Hepatitis virus A, B and C, West Nile virus, poliovirus, rhinovirus, HIV, Herpes simplex virus 1 and 2 (including encephalitis, neonatal and genital forms), human papilloma virus, cytomegalovirus, Epstein Barr virus, Hepatitis virus A, B and C, rotavirus, norovirus, adenovirus, influenza virus including influenza A virus, respiratory syncytial virus, varicella-zoster virus, small pox, monkey pox and SARS virus.

Additional examples of fungi that may be harvested and/or manipulated according to the invention include candidiasis, ringworm, histoplasmosis, blastomycosis, paracoccidioidomycosis, crytococcosis, aspergillosis, chromomycosis, mycetoma, pseudallescheriasis and tinea versicolor.

Additional examples of parasites that may be harvested and/or manipulated according to the invention include both protozoa and nematodes such as amebiasis, Trypanosoma cruzi, Fascioliasis (e.g., Facioloa hepatica), Leishmaniasis, Plasmodium (e.g., P. falciparum, P. knowlesi, P. malariae) Onchocerciasis, Paragonimiasis, Trypanosoma brucei, Pneumocystis (e.g., Pneumocystis carinii), Trichomonas vaginalis, Taenia, Hymenolepsis (e.g., Hymenolepsis nana), Echinococcus, Schistosomiasis (e.g., Schistosoma mansoni), neurocysticercosis, Necator americanus and Trichuris trichiura, Giardia.

Additional examples of mycobacteria that may be harvested and/or manipulated according to the invention include M. tuberculosis and M. leprae.

The foregoing lists of infections are not intended to be exhaustive but rather exemplary.

### Nucleic Acids

Detection and optionally identification and/or quantification of microorganisms of a sample may require the isolation of nucleic acids from the microorganisms and/or samples. In some embodiments, nucleic acids (e.g., nucleic acid fragments) can be prepared from existing nucleic acid sequences (e.g., genomic or cDNA) using molecular biology techniques, such as those employing endonucleases. Nucleic acids prepared in this manner may be referred to as isolated nucleic acids. An isolated nucleic acid generally refers to a nucleic acid that is separated from components with which it normally associates in nature. As an example, an isolated nucleic acid may be one that is separated from a cell, from a nucleus, from mitochondria, or from chromatin. The nucleic acids may be naturally occurring or non-naturally occurring nucleic acids. Non-naturally occurring nucleic acids include but are not limited to bacterial artificial chromosomes (BACs) and yeast artificial chromosomes (YACs). The term “nucleic acid” refers to multiple linked nucleotides (e.g., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and may refer to oligoribonucleotides as well as oligodeoxyribonucleotides. The terms may also include polynucleosides (e.g., a polynucleotide minus a phosphate) and any other organic base containing nucleic acid. The organic bases include adenine, uracil, guanine, thymine, cytosine and inosine.

In some embodiments, the nucleic acid is DNA or RNA. DNA includes genomic DNA (such as nuclear DNA and mitochondrial DNA), as well as in some instances complementary DNA (cDNA). RNA includes messenger RNA (mRNA), ribosomal RNA (rRNA), microRNA (miRNA), and the like. Harvest and isolation of nucleic acids are routinely performed in the art and methods that may be used in accordance with the invention can be found in standard molecular biology textbooks. (See, e.g., Sambrook et al., “Molecular Cloning: A Laboratory Manual” (2nd. Ed.), Vols. 1-3, Cold Spring Harbor Laboratory Press (1989); F. Ausubel et al., eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981)).

In accordance with the invention, the nucleic acid molecules can be directly harvested and isolated from a biological sample (such as a bodily tissue or fluid sample, or a cell culture sample) without the need for prior amplification using techniques such as polymerase chain reaction (PCR). Harvest and isolation of nucleic acid molecules are routinely performed in the art and suitable methods can be found in standard molecular biology textbooks (e.g., such as Maniatis' Handbook of Molecular Biology). Accordingly, the nucleic acid may be a non-in vitro-amplified nucleic acid. As used herein, a “non-in vitro-amplified nucleic acid” refers to a nucleic acid that has not been amplified in vitro using techniques such as polymerase chain reaction or recombinant DNA methods prior to manipulation, detection and/or analysis by the methods contemplated by the invention. A non-in vitro-amplified nucleic acid may, however, be a nucleic acid that is amplified in vivo (in the biological sample from which it was harvested) as a natural consequence of the development of the cells in vivo. This means that the non-in vitro nucleic acid may be one which is amplified in vivo as part of for example locus amplification, which is commonly observed in some cell types as a result of mutation or cancer development.

As used herein, “linked” or “linkage” means two entities bound to one another by any physicochemical means. Any linkage known to those of ordinary skill in the art, covalent or non-covalent, is embraced. Natural linkages are those ordinarily found in nature connecting for example naturally occurring entities. Natural linkages include, for instance, amide, ester and thioester linkages. Nucleic acids of the invention may comprise synthetic or modified linkages.

Nucleic acids commonly have a phosphodiester backbone because this backbone is most common in vivo. Nonetheless, nucleic acids are not so limited. Backbone modifications are known in the art, and nucleic acids having such backbone modifications are contemplated by the present invention. One of ordinary skill in the art is capable of preparing such nucleic acids without undue experimentation. Any probes, described elsewhere herein, if nucleic acid in nature, may also have backbone modifications such as those described herein.

Thus, the nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

The nucleic acids may be double-stranded, although in some embodiments, the nucleic acid targets may be denatured and presented in a single-stranded form. This may be accomplished by modulating the environment of a double-stranded nucleic acid including singly or in combination increasing temperature, decreasing salt concentration, and the like. Methods of denaturing nucleic acids are known in the art, any of which may be used herein.

Sample Pre-Treatment(s)

Certain methods provided herein are designed to be used in conjunction with automated reactors. For such reactors to function properly, the sample injected into the reactor must meet certain requirements of sample purity to avoid, for example, clogging fluidic pathways, interfering with flowstreams, and changing membrane resistance. Prior to depositing a sample in the automated reactor, the sample may be pre-treated to remove any matrices (e.g., various sized particulate).

Examples of pre-treatment steps for use in accordance with the invention include, without limitation, sedimentation (e.g., selective sedimentation using density gradient centrifugation) and/or filtration, as described in more detail below. Pretreatment protocols involve a series of some of the steps described below. Choice of pretreatment steps may depend on the type of the sample (e.g., food sample or stool sample). Generally, pretreatment protocols described here include the separation of microorganisms from matrix based on size and density. “Matrix” herein refers to cellular and extracellular debris that may be present in sample whether that sample comprises microorganisms or other cell types.

In some embodiments, a sample may be diluted in broth, buffer or water (e.g., 1 to 10 w/v or v/v with or without density gradient media and incubated at a temperature of about 35° C. to about 42° C. for about 18 to about 24 hours. Any selective or non-selective broth may be used in accordance with the invention. Non-limiting examples of broth that may be used herein include buffered peptone water (BPW), TT broth (Hajna), Modified Rappaport Vassiliadis (mRV) broth, Rappaport-Vassiliadis R10 broth, Rappaport-Vassiliadis Soya Peptone Broth (RVS), trypticase soy broth (TSB) or tryptose broth, Luria-Bertani broth, and lysogeny broth.

Sedimentation of Matrix by Gravity.

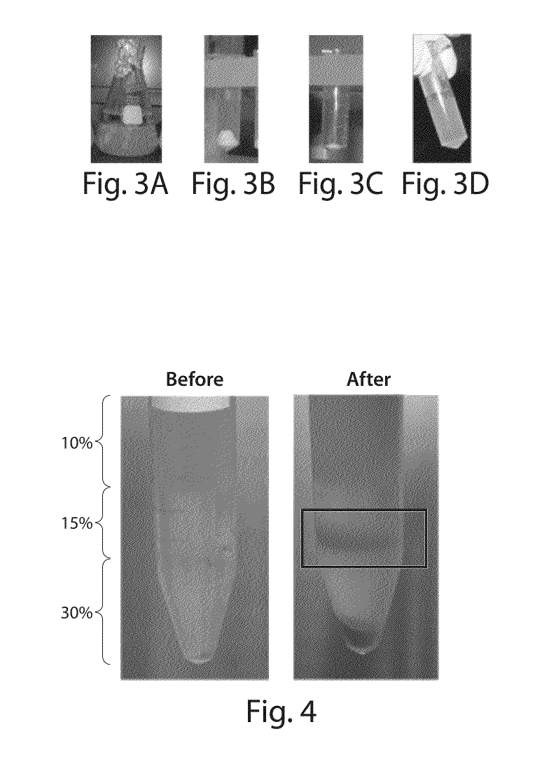

Overnight incubation permits large heavy particulates (e.g., ground meat particulates, fecal matter) to settle by gravity and low density particulates (e.g., fat in ground meat samples) to float to the top. A sample may be incubated overnight with shaking. The flask may then be removed from the shaker and permitted to stand for 30 min. A pipette may then be used to remove 2 ml of the overnight culture, avoiding large particulates (e.g., from the center of the flask), and the 2 ml of culture may be dispensed in a 2 ml vial (as shown in FIG. 3A).

Selective Sedimentation.

Selective sedimentation includes a slow spin and a fast spin. A bench top centrifuge may be used to spin 0.5-2.0 ml of sample suspension (e.g., overnight culture) at 15-30 relative centrifugal force (rcf) for 5-30 minutes at room temperature (˜20° C. to 25° C.). This slow centrifugation step pellets large particulate but not bacterial cells, which remain in the supernatant (FIG. 3B). The supernatant may be transferred to a fresh 2 ml vial and the vial with the pellet discarded. A bench-top centrifuge may then be used to spin the vial with the supernatant at 13000 rcf for 2-15 minutes at room temperature. This fast centrifugation step pellets bacterial cells and fungi but not small molecules (FIG. 3C). The supernatant may be discarded.

Centrifugation and Washing.

The pellet of bacterial cells or fungi may be resuspended in 2 ml of water, buffer or broth by pipetting. The cells may be spun down by centrifugation at 13000 rcf for 2-3 minutes at room temperature. The supernatant may be discarded. The wash procedure may be repeated 1-3 times, depending on the sample type.

Density Gradient Centrifugation: Stool Sample Example.

0.5-1.0 grams of stool sample may be resuspended in 2-6 ml of buffer or broth optionally containing 10% HistoDenz™. Alternatively, a food sample may be resuspended in 2-6 ml of buffer or broth optionally containing 10% HistoDenz™. The entire volume of sample may be layered onto a step gradient in 15 ml Falcon tube that includes 0.5-2 ml of 15% HistoDenz™ in buffer or broth and 1-2 ml of 30% HistoDenz™ in buffer or broth (i.e., a 10-15-30 gradient). Buffers may have low or high salt, may be with or without non-ionic surfactants, and may be with or without chelating agents. Broths may be selective or non-selective. HistoDenz™ gradients may be, without limitation, 10-20-40, 10-15-40 or 10-20-30, or more generally [(1.000-1.052 g/ml)-(1.078 to 1.105 g/ml)-(1.159-1.212 g/ml)] at 20° C. Density gradient compounds may be, without limitation, polysaccharides (e.g., Ficoll™), colloidal silica (e.g., Percoll®) or iodinated media (e.g., HistoDenz™). Using a centrifuge equipped with a swing-bucket rotor, the tubes may be centrifuged at 3200 rcf for 40-90 minutes at 4° C. 1-2 ml of the culture at the 15% to 30% interface may be recovered or, when using gradients with 20% in the middle layer, the entire 20% layer plus both interfaces may be recovered in a total volume of 2 ml (FIG. 4).

Density Gradient Centrifugation: Mold Culture Example.

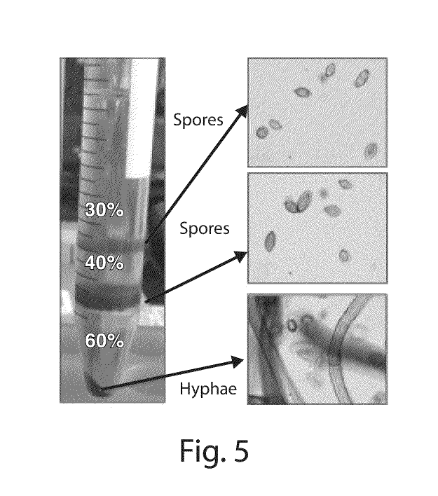

0.5-2 ml of mold culture having an optical density (OD) of 2-20 may be resuspended in 2-6 ml of buffer or broth optionally containing 10% HistoDenz™. The entire volume may be layered onto a step gradient in a 15 ml Falcon tube that includes 0.5-2 ml of 30% HistoDenz™ in buffer or broth, 0.5-2 ml of 40% HistoDenz™ in buffer or broth, and 0.5-2 ml of 60% HistoDenz™ in buffer or broth (i.e., 10-30-40-60 gradient). HistoDenz™ gradients may be, without limitation, 10-20-40-60 or 10-30-70, or more generally [(1.000-1.052 g/ml)-(1.105 to 1.265 g/ml)-(1.319-1.426 g/ml)] at 20° C. Density gradient separation may be performed as described above. 1-2 ml of one or more band(s) formed in the middle of the vial may be recovered (FIG. 5). Density gradients may be different for different molds, depending on the physical properties of their hyphae.

The above volumes may be decreased by a factor of 5-10 for density gradient centrifugation in a 2 ml vial tube using a bench top centrifuge. Centrifugation may be performed at 13000 rcf for 5-10 minutes at room temperature. This treatment (i) decreases the time of pre-treatment, (ii) utilizes bench top equipment but (iii) decreases the volume of sample to be processed, and (iv) decreases the microorganism load.

Filtration.

1-5 ml of a sample suspension in buffer or broth, or a 1-2 ml fraction may be recovered from the density gradient centrifugation and filtered through a 100 μm or 50 μm steel filter. The filter may be washed with 1-4 ml of buffer or broth. The combined flow-through may be optionally further filtered through a 20 μm or 10 μm nylon mesh filter. The mesh filter may be washed with 1-4 ml of buffer or broth. Centrifugation of the flow-through at 3200 rcf for 40 minutes at 4° C., or at 13000 rcf for 5 minutes at room temperature, results in a bacterial pellet.

Preparation of Cell Suspension for Injection.

The pellet may be resuspended in water, buffer or broth to a concentration corresponding to 0.1 to 2 OD (e.g., OD=1) for bacterial cells or 2-20 OD (e.g., OD=10) for fungi (e.g., mold). The cell suspension is then ready for injection into an automated reactor (FIG. 3D).

pH Adjustments: —

Growth of microorganisms in selective enrichment broths leading to acidification of the broth (e.g., E. coli in MacConkey broth) can result in precipitation of media ingredients. In such cases, further treatment of the sample may occur to prevent carry-over of particulate matter to the membrane reactor. Adjustment of pH of the sample, after removal of the coarse particle by slow centrifugation at 15-30 relative centrifugal force (rcf) for 5-30 minutes, to neutral pH by adding 1 part of a buffer (e.g., 1M Tris chloride, pH 7.6) to 10-15 parts of the sample may be done to dissolve the precipitate.

### Lytic Step(s)

The number of and order of the lytic step(s) may be adjusted to target one or more microorganisms or mixtures of microorganisms. The number of and order of the lytic step(s) may also be adjusted based on the type of microorganism. For example, particular lytic reagents and chemistries may be based on the structure and chemistry of the particular microorganism. Additionally, some of the lytic reagents can act together in a single reaction (e.g., lysozyme and achromopeptidase), while others may require a separate reaction (e.g., proteinase K).

Lytic reagents are well-known in the field and may include, without limitation lysozyme, mutanolysin, lysostaphin, labiase, achromopeptidase, lyticase and/or proteinase K (see Niwa, T et al. J Biol Methods, 61:251-260, 2005; Ezaki, T and Suzuki, S J Clin Microbiology, 16:844-846, 1982; Zhong, W. et al. Appl Environmental Microbiol, 73:3446-3449, 2007; Halpin, J L et al. Foodborne Pathogens and Disease, 7:293-298, 2010 and Ribot, E M et al. Foodborne Pathogens and Disease. 3:59-67, 2006). In some embodiments, about 25 μg to about 500 μg of lytic enzyme may be used in a lytic step of the invention. For example, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 150 μg, about 200 μg, about 250 μg, about 300 μg, about 350 μg, about 400 μg, about 450 μg or about 500 μg of lytic enzyme may be used.

In some embodiments, about 50 to about 500 units of lytic enzyme may be used in a lytic step of the invention. For example, about 50 units, about 55 units, about 60 units, about 65 units, about 70 units, about 75 units, about 80 units, about 85 units, about 90 units, about 95 units, about 100 units, about 150 units, about 200 units, about 250 units, about 300 units, about 350 units, about 400 units, about 450 units or about 500 units of lytic enzyme may be used.

The choice of lytic reagents, including lytic enzyme, may also depend on the number of types of microorganisms present in a sample (or expected to be present in the sample). For example, the lytic reagents may include: proteinase K if the microorganism is a virus; lysozyme and proteinase K if the microorganism is Escherichia coli (E. coli); lysozyme, lysostaphin and proteinase K if the microorganisms are E. coli and Staphylococcus spp; lysozyme, lyticase and proteinase K if the microorganism is yeast; lysozyme, lysostaphin, lyticase and proteinase K if the microorganisms are E. coli, Staphylococcus spp and yeast.

In some embodiments, the lytic reagents may comprise denaturing agents (e.g., urea), detergents (e.g., sodium dodecyl sulfate (SDS), polysorbate (e.g., Tween® 20), Triton™ X-100), chelating agents (e.g., ethylenediaminetetraacetic acid (EDTA)), reducing agents (e.g., beta-mercaptoethanol (BME)), lytic buffer solutions (e.g., tris(hydroxymethyl)aminomethane (Tris)) or any combination of two or more of the foregoing. In some embodiments, the pH of the buffer solution is about 2 to about 8. For example, the pH of a digest buffer may be about 2, 2.5, 3, 3.5, 4, 4.5, 5, 5.5, 6, 6.5, 7, 7.5 or 8.

In some embodiments, the lytic reagents may comprise one or more lytic enzymes and SDS, EDTA, BME, urea and Tris. For example, the lytic reagents may comprise one or more lytic enzymes and 0.1-1% w/v SDS, 1-50 mM EDTA, 0-2% v/v BME, 0-7 M urea and 10-50 mM Tris, pH 6-8. In some embodiments, the lytic reagents may comprise one or more lytic enzymes and Tween® 20, Triton™ X-100, EDTA and Tris. For example, the lytic reagents may comprise one or more lytic enzymes and 0.1-1% Tween® 20, 0.1-1% Triton™ X-100, 10-50 mM EDTA and 10-50 mM Tris, pH 6-8.

A lytic step may be carried out for a set period of time. For example, a sample may be incubated with lytic reagents for about 5 minutes to about an hour. In some embodiments, a sample may be incubated with lytic reagents for about 5, about 10, about 15, about 20, about 25, about 30, about 35, about 40, about 45, about 50, about 55 or about 60 minutes. Multiple lytic steps may have different incubation periods.

A lytic step may be carried out at a set temperature. For example, a sample may be incubated with lytic reagents at a temperature of about 37° C. to about 75° C. In some embodiments, a sample may be incubated with lytic reagents at a temperature of about 37° C. to about 65° C., 37° C. to about 55° C., 37° C. to about 45° C. In some embodiments, a sample may be incubated with lytic reagents at a temperature of 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C. or 75° C. Multiple lytic steps may have different incubation temperatures.

A lytic step may be carried out once, twice, three times, four times or five times using the same sample, for example, in the same reaction chamber. If a lytic step is carried out more than once, the lytic agents used in each step may differ from each other. For example, in embodiments, where two lytic steps are carried out, lysostaphin may be used in the first step as the lytic enzyme in combination with a suitable reagents such as Tween® 20, Triton™ X-100, EDTA and Tris; and proteinase K may be used in the second step as the lytic enzyme in combination with a suitable reagents such as SDS, EDTA, BME, urea and Tris.

### Digest Step(s)

The choice of digest reagents (e.g., endonuclease(s)) for use in accordance with the invention may depend on the method of analysis. For example, a particular method may require endonucleases that produce a certain number and size of nucleic acid fragments. As examples, PFGE analysis typically requires DNA fragments between 20 kb and 800 kb; and GSS analysis typically requires DNA fragments between 80 kb and 350 kb.

The choice of digest reagents may also be based on the GC content of the nucleic acid of the microorganisms in the sample (or those microorganisms expected to be in the sample). For example, a combination of digest reagents (e.g., endonucleases and/or buffers) may be required the microorganisms contains species with wide ranges of GC content. For example, the digest reagents may include ApaI and XbaI if the microorganisms are E. coli, Pseudomonas spp and Staphylococcus spp. In this case, the two endonucleases should be applied to the nucleic acid as two separate digest reactions (e.g., one endonuclease per digest reaction), rather than a single reaction containing both endonucleases.

Further, the choice of digest reagents may depend on the number of types of microorganisms present in a sample (or expected to be present in the sample). For example, the digest reagents may include: ApaI or XbaI if the microorganism is E. coli (˜50%); ApaI if the microorganisms are E. coli and Staphylococcus spp (˜30-33%); or XbaI if the microorganisms are E. coli and Pseudomonas spp (˜60-66%).

Endonucleases that may be used in accordance with the invention include, without limitation, AatII, Acc65I, AccI, AciI, AclI, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvaII, AvrII, BaeI, BamHI, BanI, BanII, BbsI, BbvCI, BbvI, BccI, BceAI, BcgI, BciVI, BclI, BfaI, BfuAI, BfuCI, BglI, BglII, BlpI, Bme1580I, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSI, BstAPI, BstBI, BstEII, BstNI, BstUI, BstXI, BstYI, BstZ17I, Bsu36I, BtgI, BtgZI, BtsCI, BtsI, Cac8I, ClaI, CspCI, CviAII, CviKI-1, CviQI, DdeI, DpnI, DpnhI, DraI, DraIII, DrdI, EaeI, EagI, EarI, EciI, EcoNI, EcoO109I, EcoRI, EcoRV, FatI, FauI, Fnu4HI, FokI, FseI, FspI, HaeII, HaeIII, HgaI, HhaI, HincII, HindIII, HinfI, HinP1I, HpaI, HpaII, HphI, Hpy188I, Hpy188III, Hpy99I, HpyAV, HpyCH4III, HpyCH4IV, HpyCH4V, KasI, KpnI, MboI, MboII, MfeI, MluI, MlyI, MmeI, MnlI, MscI, MseI, MslI, MspA1I, MspI, MwoI, NaeI, NarI, NciI, NcoI, NdeI, NgoMIV, NheI, NlaIII, NlaIV, NmeAIII, NotI, NruI, NsiI, NspI, PacI, PaeR7I, PciI, PflFI, PflMI, PhoI, PleI, PmeI, PmlI, PpuMI, PshAI, PsiI, PspGI, PspOMI, PspXI, PstI, PvuI, PvuII, RsaI, RsrII, SacI, SacII, SalI, SapI, Sau3AI, Sau96I, SbfI, ScaI, ScrFI, SexAI, SfaNI, SfcI, SfiI, SfoI, SgrAI, SmaI, SmlI, SnaBI, SpeI, SphI, SspI, StuI, StyD4I, StyI, SwaI, TaqI, TfiI, TliI, TseI, Tsp45I, Tsp509I, TspMI, TspRI, Tth111I, XbaI, XcmI, XhoI, XmaI, XmnI, and ZraI.

In some embodiments, about 1 μg to about 100 μg of endonuclease may be used in a digest step of the invention. For example, about 1 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg or about 100 μg of endonuclease may be used.

In some embodiments, about 1 unit to about 100 units of endonuclease may be used in a digest step of the invention. For example, about 1 unit, about 5 units, about 10 units, about 15 units, about 20 units, about 25 units, about 30 units, about 35 units, about 40 units, about 45 units, about 50 units, about 55 units, about 60 units, about 65 units, about 70 units, about 75 units, about 80 units, about 85 units, about 90 units, about 95 or about 100 units of endonuclease may be used.

In some embodiments, the digest reagents may comprise buffer solutions that contain potassium acetate, Tris-acetate, magnesium acetate, NaCl, Tris-HCl, MgCl₂, Bis-Tris-Propane-HCl, dithiothreitol (DTT), bovine serum albumin

In some embodiments, the digest buffer may contain Tris-Propane-HCl, MgCl₂ and DTT. For example, the digest buffer may contain 10 mM Bis-Tris-Propane-HCl, 10 mM MgCl₂, and 1 mM DTT. In some embodiments, the digest buffer may contain NaCl, Tris-HCl, MgCl₂ and DTT. For example, the digest buffer may contain 50 mM NaCl, 10 mM Tris-HCl, 10 mM MgCl₂ and 1 mM DTT, or the digest buffer may contain 100 mM NaCl, 50 mM Tris-HCl, 10 mM MgCl₂ and 1 mM DTT. In some embodiments, the digest buffer contains potassium acetate, Tris-acetate, magnesium acetate and DTT. For example, the digest buffer may contain 50 mM potassium acetate, 20 mM Tris-acetate, 10 mM magnesium acetate and 1 mM DTT.

In some embodiments, the pH of the buffer is about 2 to about 8. For example, the pH of a digest buffer may be about 2, 2.5, 3, 3.5, 4, 4.5, 5, 5.5, 6, 6.5, 7, 7.5 or 8. In some embodiments, the pH of the buffer is 7.9. In some embodiments, the pH of the buffer is 7.0.

A digest step may be carried out for a set period of time. For example, a sample may be incubated with digest reagents for about 5 minutes to about an hour. In some embodiments, a sample may be incubated with lytic reagents for about 5, about 10, about 15, about 20, about 25, about 30, about 35, about 40, about 45, about 50, about 55 or about 60 minutes. Multiple digest steps may have different incubation periods.

A digest step may be carried out at a set temperature. For example, a sample may be incubated with lytic reagents at a temperature of about 20° C. to about 42° C. In some embodiments, a sample may be incubated with lytic reagents at a temperature of 20° C. ° C., 21° C. ° C., 22° C. ° C., 23° C., 24° C., 25° C., 26° C., 27° C., 27° C., 29° C., 30° C., 21° C., 32° C., 33° C., 34° C., 35° C., 36° C., 37° C., 38° C., 39° C., 40° C., 41° C. or 42° C. Multiple digest steps may have different incubation temperatures.

A digest step may be carried out once, twice, three times, four times or five times using the same sample, for example, in the same reaction chamber. If a digest step is carried out more than once, the digest agents used in each step may differ from each other. For example, in embodiments, where two digest steps are carried out, ApaI may be used in the first step as the endonuclease in combination with a suitable buffer that includes, for example, potassium acetate, Tris-acetate, magnesium acetate and DTT; and AgeI may be used in the second step as the endonuclease in combination with a suitable buffer that includes, for example, Tris-Propane-HCl, MgCl₂ and DTT.

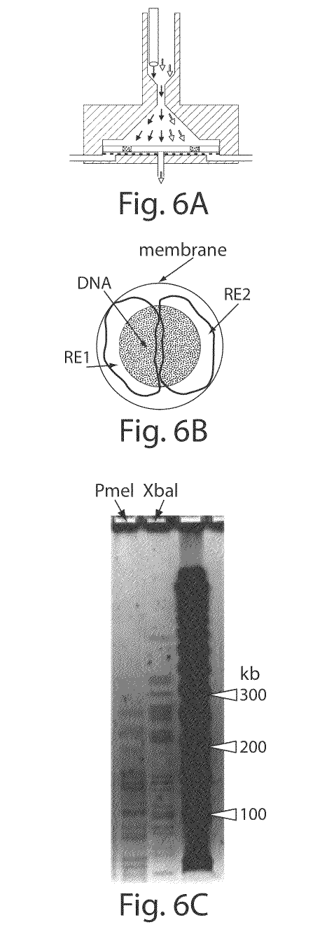

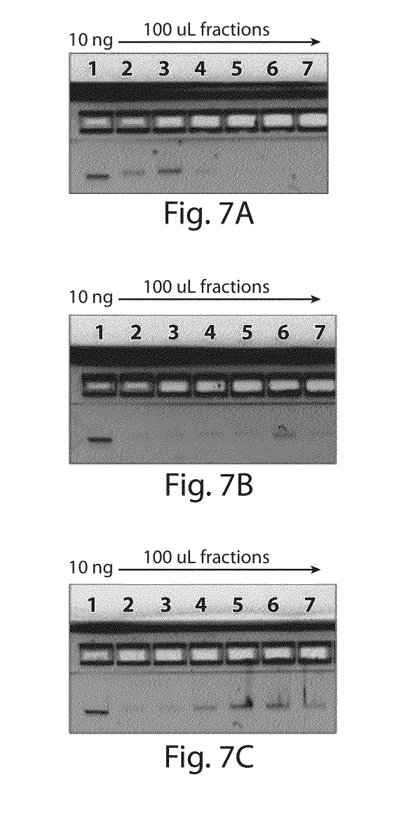

In some embodiments, two independent restriction digests can be carried out in an automated reaction chamber. Such a process typically begins with a single DNA population and yields two populations therefrom: one that is digested with a first enzyme and one that is digested with a second enzyme. The process is now described briefly. A sample is first deposited through a fluid port and onto a porous substrate (e.g., membrane such as an ultrafiltration membrane) in the chamber. A fluid containing lytic reagents is then deposited through the fluid port and onto the porous substrate in the chamber, and the porous substrate is incubated at a set temperature for a period of time. This step may be repeated, if necessary. At the end of the lytic incubation period, an endonuclease buffer solution is passed through the fluid port and onto the substrate to wash away the lytic reagents. Next, an endonuclease buffer solution containing a first endonuclease (“RE1”) is deposited through the fluid port off-center (FIG. 6A) onto the left half of the porous substrate (FIG. 6B) and permitted to incubate at a set temperature for a set period of time. The digested nucleic acid on the left half of the substrate is then eluted back up through the fluid port, leaving undigested on the right half of the substrate.

A second endonuclease buffer solution is then passed through the fluid port. Next, an endonuclease buffer solution containing a second endonuclease (“RE2”) is deposited through the fluid port off-center onto the right half of the porous substrate (FIG. 6B) and permitted to incubate at a set temperature for a set period of time. The digested nucleic acid on the right half of the substrate is then eluted back up through the fluid port. This double digestion reaction yields two nucleic acid digests of the same sample, which then may be analyzed separately or combined for further analysis (FIG. 6C).

### Tagging Step(s) and Probes

The methods provided herein involve the use of a probe that binds to the nucleic acid being studied in a sequence-specific manner. A probe is a molecule that specifically recognizes and binds to particular sequences within a nucleic acid in a sequence-specific manner.

Binding of a probe to a nucleic acid indicates the presence and location of a sequence in the target nucleic acid that is complementary to the sequence of the probe, as will be appreciated by those of ordinary skill in the art. As used herein, a polymer that is bound by a probe is “labeled” with the probe. The position of the probe along the length of a target polymer indicates the location of the complementary sequence in the polymer.

The probe may itself be a polymer but it is not so limited. Examples of suitable probes are nucleic acids and peptides and polypeptides and peptide nucleic acids (PNAs) including bis-PNAs. As used herein a “peptide” is a polymer of amino acid residues connected preferably but not solely with peptide bonds. Other probes include but are not limited to sequence-specific major and minor groove binders and intercalators, nucleic acid binding peptides or polypeptides, etc.

The probes can include nucleotide derivatives such as substituted purines and pyrimidines (e.g., C-5 propyne modified bases (Wagner et al., 1996, Nature Biotechnology, 14:840-844)). Suitable purines and pyrimidines include but are not limited to adenine, cytosine, guanine, thymidine, pseudoisocytosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. The probes can also include non-naturally occurring nucleotides, or nucleotide analogs. Other such modifications are known to those of skill in the art.

The probes also encompass substitutions or modifications, such as in the bases and/or sugars. For example, they include nucleic acid molecules having backbone sugars which are covalently attached to low molecular weight organic groups other than a hydroxyl group at the 3′ position and other than a phosphate group at the 5′ position. Thus, modified nucleic acid molecules may include a 2′-O-alkylated ribose group. In addition, modified nucleic acid molecules may include sugars such as arabinose instead of ribose. Thus, the probes may be heterogeneous in composition at both the base and backbone level. In some embodiments, the probes are homogeneous in backbone composition (e.g., all phosphodiester, all phosphorothioate, all peptide bonds, etc.).

The probe may be of any length, as can the sequence to which it binds. In instances in which the polymer and the probe are both nucleic acid molecules, the length of the probe and the sequence to which it binds are generally the same. The length of the probe will depend upon the particular embodiment. The probe may range from at least 4, at least 5, at least 6, at least 7, at least 8, at least 9, at least 10, at least 12, at least 15, at least 20, at least 25, at least 50, at least 75, at least 100, at least 150, at least 200, at least 250, at least 500, or more nucleotides (including every integer therebetween as if explicitly recited herein). Preferably, the probes are at least 8 nucleotides in length to in excess of 1000 nucleotides in length.

In some embodiments, shorter probes are more desirable because they provide much sequence information leading to a higher resolution sequence map of the target nucleic acid molecule. Longer probes are desirable when unique gene-specific sequences are being detected. The length of the probe however determines the specificity of binding. Proper hybridization of small sequences is more specific than is hybridization of longer sequences because the longer sequences can embrace mismatches and still continue to bind to the target depending on the conditions. One potential limitation to the use of shorter probes however is their inherently lower stability at a given temperature and salt concentration. In order to avoid this latter limitation, bisPNA or two-arm PNA probes can be used which allow both shortening of the probe and sufficient hybrid stability in order to detect probe binding to the target nucleic acid molecule.

In some instances, nucleic acid probes will form at least a Watson-Crick bond with a target nucleic acid. In other instances, the nucleic acid probe can form a Hoogsteen bond with the target nucleic acid, thereby forming a triplex. A nucleic acid probe that binds by Hoogsteen binding enters the major groove of a nucleic acid polymer and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid polymer. BisPNA probes, for instance, are capable of both Watson-Crick and Hoogsteen binding to a nucleic acid.

In some embodiments, the probe is a nucleic acid that is a peptide nucleic acid (PNA), a bisPNA clamp, a pseudocomplementary PNA, a locked nucleic acid (LNA), DNA, RNA, or co-nucleic acids of the above such as DNA-LNA co-nucleic acids. siRNA or miRNA or RNAi molecules can be similarly used.

In some embodiments, the probe is a peptide nucleic acid (PNA), a bisPNA clamp, a locked nucleic acid (LNA), a ssPNA, a pseudocomplementary PNA (pcPNA), a two-armed PNA (as described in U.S. Publication No. 2003-0215864 A1 (published Nov. 20, 2003) and International Publication No. WO 03/091455 A1 (published Nov. 6, 2003)), or co-polymers thereof (e.g., a DNA-LNA co-polymer).

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes. BisPNA includes two strands connected with a flexible linker. One strand is designed to hybridize with DNA by a classic Watson-Crick pairing, and the second is designed to hybridize with a Hoogsteen pairing. Pseudocomplementary PNA (pcPNA) (Izvolsky, K. I. et al., Biochemistry 10908-10913 (2000)) involves two single stranded PNAs added to dsDNA. Locked nucleic acid (LNA) molecules form hybrids with DNA, which are at least as stable as PNA/DNA hybrids (Braasch, D. A. et al., Chem & Biol. 8(1):1-7(2001)).

The probes are preferably single-stranded, but they are not so limited. For example, when the probe is a bisPNA it can adopt a secondary structure with the nucleic acid polymer resulting in a triple helix conformation, with one region of the bisPNA clamp forming Hoogsteen bonds with the nucleotide bases of the polymer and another region of the bisPNA clamp forming Watson-Crick bonds with the nucleotide bases of the polymer.

Sequence-specific probes used in Genome Sequence Scanning™ (GSS™) may be bis-peptide nucleic acids (bisPNAs). bisPNAs may consist of two strands of 6- to 8-mer PNAs connected via flexible linker and fluorescent dye attached to one of the ends through a flexible linker. bisPNAs may be complementary to a 6 to 8 base-long DNA sequence consisting of adenines and guanines. The frequency of bisPNA binding to genomic DNA depends on GC content of target DNA and the sequence of bisPNA: optimal frequency is about a dozen complimentary sites per 100 kb of genomic DNA. The sequence of bisPNAs may be chosen to target microorganisms with different GC contents.

The probes of the invention may be labeled with detectable molecules. As used herein, the terms “detectable molecules” and detectable labels” are used interchangeably. The detectable molecule may be detected directly, for example, by its ability to emit and/or absorb light of a particular wavelength. Alternatively, a molecule may be detected indirectly, for example, by its ability to bind, recruit and, in some cases, cleave another molecule which itself may emit or absorb light of a particular wavelength. An example of indirect detection is the use of an enzyme which cleaves an exogenously added substrate into visible products. The label may be of a chemical, peptide or nucleic acid nature although it is not so limited. When two or more detectable molecules are to be detected, the detectable molecules should be distinguishable from each other. This means that each emits a different and distinguishable signal from the other.

Detectable molecules may be conjugated to probes using chemistry that is known in the art. The labels may be directly linked to the DNA bases or may be secondary or tertiary units linked to modified DNA bases. Labeling with detectable molecules may be carried out either prior to or after binding to a target nucleic acid molecule. In some embodiments, a single nucleic acid molecule is bound by several different probes at a given time and thus it is advisable to label such probes prior to target binding. Labeled probes are also commercially available.

Generally, the detectable molecule may be selected from the group consisting of an electron spin resonance molecule (such as for example nitroxyl radicals), a fluorescent molecule, a chemiluminescent molecule, a radioisotope, an enzyme substrate, a biotin molecule, an avidin molecule, a streptavidin molecule, an electrical charged transducing or transferring molecule, a nuclear magnetic resonance molecule, a semiconductor nanocrystal or nanoparticle, a colloid gold nanocrystal, an electromagnetic molecule, a ligand, a microbead, a magnetic bead, a paramagnetic particle, a quantum dot, a chromogenic substrate, an affinity molecule, a protein, a peptide, a nucleic acid molecule, a carbohydrate, an antigen, a hapten, an antibody, an antibody fragment, and a lipid.

Specific examples of detectable molecules include fluorescent labels and dyes such as those having a high extinction coefficient, high fluorescence quantum yield and high photostability (e.g., ATTO 390, ATTO 425, ATTO 465, ATTO 488, ATTO 495, ATTO 514, ATTO 520, ATTO 532, ATTO RhoGC, ATTO 550, ATTO 565, ATTO Rho3B, ATTO Rho11, ATTO Rho12, ATTO Thio12, ATTO Rho101, ATTO 590, ATTO 594, ATTO Rho13, ATTO 633, ATTO 647, ATTO 647N, ATTO 655, ATTO Oxa12, ATTO 665, ATTO 680, ATTO 700, ATTO 725 and ATTO 740 commercially available from ATTO-TEC, Denmark). Other examples of detectable molecules include radioactive isotopes such as P³² or H³, fluorophores such as fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as alkaline phosphatase, horseradish peroxidase, β-galactosidase, and hapten conjugates such as digoxigenin or dinitrophenyl, etc. Other detectable markers include chemiluminescent and chromogenic molecules, optical or electron density markers, etc. The probes can also be labeled with semiconductor nanocrystals such as quantum dots (i.e., Qdots), described in U.S. Pat. No. 6,207,392. Qdots are commercially available from Quantum Dot Corporation. Bis-PNA probes may be labeled with ATTO dyes such as those available from ATTOtec.

In some embodiments, the probes are labeled with detectable molecules that emit distinguishable signals detectable by one type of detection system. For example, the detectable molecules can all be fluorescent labels or radioactive labels. In other embodiments, the probes are labeled with molecules that are detected using different detection systems. For example, one probe may be labeled with a fluorophore while another may be labeled with radioactive molecule.

Analysis of the nucleic acid involves detecting signals from the detectable molecules, and determining their position relative to one another. In some instances, it may be desirable to further label the target nucleic acid molecule with a standard marker that facilitates comparison of information obtained from different targets. For example, the standard marker may be a backbone label, or a label that binds to a particular sequence of nucleotides (be it a unique sequence or not), or a label that binds to a particular location in the nucleic acid molecule (e.g., an origin of replication, a transcriptional promoter, a centromere, etc.).

One subset of backbone labels are nucleic acid stains that bind nucleic acid molecules in a sequence independent or sequence non-specific manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO (e.g., PO-PRO-1, PO-PRO-3), BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

It is to be understood that the labeling of the probe should not interfere with its ability to recognize and bind to a nucleic acid molecule.

In some embodiments, an analysis intends to detect preferably two or more detectable signals. As described herein, a first probe can interact with the energy source to produce a first signal and a second probe can interact with the energy source to produce a second signal. The signals so produced may be different from one another, thereby enabling more than one type of unit to be detected on a single target polymer. In some instances, two or more probes may be used that are identically labeled. In some instances, two or more probes may be used that are differentially labeled. In some instances, two or more probes may be used, each differentially labeled from all the other probes. In some instances, two or more probes may be used in which first subset of probes is identically labeled and a second subset of probes is identically labeled but wherein the first and the second subsets are differentially labeled relative to each other. Typically, whether identically or differentially labeled, probes will have differing binding specificity. Use of detection molecules that emit distinct signals (e.g., one emits at 535 nm and the other emits at 630 nm) enables more thorough sequencing of a target polymer since units located within the known detection resolution can now be separately detected and their positions can be distinguished and thus mapped along the length of the polymer.

In some embodiments, probes having more than one detectable label may be used as this may give rise to stronger signal on an individual nucleic acid target level. In some instances, the position of the detectable labels, their nature, and the method of attaching them to the probe, including distance to either arm of a bisPNA probe for example, may be important. Reference can be made to published US patent application 2012/0283955 for a discussion of these factors. Other probes for use in accordance with the invention are described in U.S. Pat. No. 8,361,716 B2, and U.S. Publication No. 2010/0120101.

In some embodiments, PNA having two or more fluorophores are useful as probes even if they lead to a greater number of peaks that do not represent a true match site.

### Additional Methods and Compositions of the Invention

The methods and/or reactors provided herein may be used for various purposes and applications such as, but not limited to, those described herein. Additionally, it is to be understood that any of the methods provided herein may be used in conjunction with any of the reactors described herein or in U.S. Pat. No. 8,361,716 B2, U.S. Publication No. 2010/0120101, U.S. Provisional Application No. 61/625,743 (filed Apr. 18, 2012), or Mollova et al. Anal Biochemistry, 391:135-143, 2009, each of which is incorporated by reference herein in its entirety, for various purposes and applications such, but not limited to, those described herein.

For example, the methods and/or reactors may be used generally in the preparation (including isolation) and/or manipulation of high molecular weight nucleic acids such as high molecular weight DNA. They may also be used for the preparation (including isolation) and/or manipulation of other molecules such as but not limited to proteins.

With respect to nucleic acid preparation and manipulation, the methods and/or reactors may be used to lyse cells (e.g., the source of the nucleic acids), to isolate and purify the nucleic acids from the cell lysate, to digest the nucleic acids using for example restriction enzymes, to hybridize probes to the nucleic acids, and optionally to stain the nucleic acids with backbone stains. It is contemplated that the methods and/or reactors may be used to perform all of these steps, or some of these steps, including any subset of steps in any order that is deemed suitable by one of ordinary skill in the art in view of the teachings provided herein. For example, the nucleic acid may be removed (eluted) from the reactor after digestion with the enzyme without hybridization to a probe and without exposure to the backbone stain. The methods and/or reactors may also be used to introduce different enzymes at the same or different times (including partially overlapping times) and/or to change buffer or other reactor components.

The methods and/or reactors may be used to hybridize the target nucleic acids to any variety of probes including DNA-based probes, or probes that are DNA-mimics such as but not limited to LNA, PNA, bisPNA, and the like.

Further, the methods and/or reactors may be used to synthesize nucleic acids including copies of the isolated nucleic acids using for example enzymatic reactions. Such reactions may include detectably labeled nucleotides intended for incorporation into the newly synthesized nucleic acid. The detectably labeled nucleotides may be fluorescently labeled. As another example, a nicking enzyme may be used to nick one strand of a double stranded DNA, following which fluorophore labeled nucleotides may be introduced into the nicked site using a polymerase such as Taq polymerase. See, for example, Lam et al Nat. Biotech. 30: 771(2012) for a description of such labeling methodology. Other labeling or hybridization schemes may also be implemented using the reactor of the invention.

It is therefore to be understood that the methods and/or reactors described herein may be used to perform virtually any biochemical manipulation or reaction that is known or that will be contemplated. Further examples of method and/or reactor use are provided below.

The methods and/or reactors may be used to effect controlled shearing of nucleic acids such as high molecular weight DNA. This may be accomplished using convectional fluid flows within the reactor in combination with high temperature where AT-rich regions of the DNA start melting (about 70° C. in the absence of extra salt). In some embodiments, a population of DNA fragments that are, for example, on the order of hundreds of kilobases (e.g., in the range of about 100 kb to about 1000 kb) may be reliably generated using the methods and/or reactor. The distribution of the lengths of the DNA fragments may be to a certain extent controlled by the time of the incubation. In this way, the population of DNA fragments represents the majority of DNA fragments in the entire composition (e.g., they may represent equal to or greater than 50%, 60%, 70%, 80%, 90%, 95% or 100% of the DNA fragments). The amount of DNA of various sizes may be measuring by measuring intensity of backbone labeling in gel electrophoretic bands (or regions) greater than and less than any particular threshold. The reliable generation of DNA fragments within this size range has been difficult using prior art methods and reactors/devices.

The ability to prepare and manipulate nucleic acids also makes the methods and/or reactors amenable to synthetic biology reactions. The construction of genomes, such as for example artificial bacteria genomes, requires the ability to conjugate DNA molecules of similar length to each other (e.g., conjugation of long DNA molecules to each other) and/or to conjugate DNA molecules of differing length to each other (e.g., conjugation of long DNA to short DNA). The difference in size between the long and short DNA may be about 10-fold, about 100-fold, about 1000-fold, or more. For example, the difference in size between the long and short DNA may be 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold or 1000-fold, or more. As an example, a DNA that is in the 10 kb range may be conjugated to a DNA that is in the 1 kb range or in the 100 kb range. This has proven to be an extremely inefficient reaction when performed using prior art methods including for example by simply mixing the nucleic acids in a test tube. Reasons for this reported inefficiency include the low concentration of DNA termini in a solution of long DNA, the loose structure of DNA coils due to its rigidity and strong charge, the difficulty in compressing DNA to achieve higher DNA density, and the tendency of one DNA to avoid penetrating the space of another DNA. The reaction rate for conjugating DNAs to each other is proportional to the second power of the concentration of the termini being conjugated to each other. As a result, conjugation of one (a “first”) long DNA to other (a “second”) long DNA or to shorter DNA is inherently a slow and inefficient process. This is the case even when termini are provided as “sticky ends.” Moreover, longer DNA is more susceptible to degradation by unintended shearing. Such shearing is difficult to avoid during the DNA and genomic assembly process. As a result, DNA loss can occur throughout a process and may be particularly acute where a multitude of reactions are required or performed.

The methods and/or reactors of the invention may be used to perform these reactions with improved DNA assembly and recovery. The methods and/or reactors overcomes some of the problems that are encountered when the reactions are carried out in a test tube or in solution. First, the methods and/or reactors allow the DNA to be compressed by flow in order to concentrate it on the membrane surface. Second, long DNA is maintained and preserved in the reactor during manipulations, thereby reducing losses that can otherwise occur through multiple reaction steps.

The methods and/or reactors may also be used to modify molecules such as proteins. A typical approach to modify a protein by attaching a reagent to it involves combining the protein with the reagent, incubating under certain conditions, and removing unreacted reagent from the mixture. Unreacted reagents are usually removed using gel filtration (such as size-exclusion chromatography) or by membrane filtration. Because the proteins are small, the filters are usually made of screen membranes that have high hydrodynamic resistance and the filtration process requires centrifuges to achieve sufficient gravity forces. Gel filtration is often also performed using centrifuge units for simplicity (although the efficiency and losses are usually higher compared to chromatographic columns).

In order to overcome these problems, proteins may be modified using the methods and/or reactors described herein. A protein may be introduced into a reactor and concentrated on the membrane surface. Reagent may then be introduced into the reactor and the mixture may be incubated at a suitable temperature and under suitable conditions. Following this incubation, the unreacted reagent may be removed either by elution through the membrane or by field filtration (e.g., removal of the reagent by tangential flow).

In these and various other manipulations and reactions, the methods and/or reactors of the invention demonstrate a variety of advantages over the methods and reactors/devices of the prior art. First, the reactor systems described herein are completely automated. They therefore avoid operator-dependent variability and error. Second, they are robust even over repeated operation and numerous reaction steps. For example, if multiple manipulations of a target are required, these are all performed without removal of the target from the reactors. The target may be held immobilized on the membrane surface and the reagents and buffers and other additives are flowed in and out of the reactor. This results in less loss of the target itself. Third, the methods and/or reactors are particularly useful and robust for applications involving large molecules such as proteins or high molecular weight DNA. Even removal of large molecules such as antibodies or other proteins types (where such proteins are not the target) is achieved more easily and more completely using the methods and/or reactors provided herein.

Accordingly, the invention contemplates a method comprising obtaining a population of nucleic acids having a size in the range of about 100 kb to about 1000 kb. These preparations include a high proportion of long DNA fragments, while the proportion of the fragments corresponding to sizes that are less than 100 kb (e.g., in the range of less than 1 kb to 50 kb, or to 60 kb, or to 70 kb, or to 80 kb, or to 90 kb, in some embodiments) is smaller. In addition, these preparations have a higher average size per fragment as compared to prior art methods. Alternatively, the nucleic acids having a size in the range of about 100 kb to about 1000 kb may represent the majority of the nucleic acids in the population or in a composition comprising the population. The majority may be more than 70%, more than 80%, more than 90%, more than 95%, more than 99% of nucleic acids in the population or composition (by weight, for example). These nucleic acids may be in the range of about 100 kb to about 200 kb, or to about 300 kb, or to about 400 kb, or to about 500 kb, or to about 600 kb, or to about 700 kb, or to about 800 kb, or to about 900 kb, or to about 1000 kb. The proportion or percentage of nucleic acids in a preparation (or population) may be expressed by the number of DNA molecules or the mass of DNA molecules having length above or below a particular threshold (and including the threshold itself, as the case may be). Mass of DNA may be determined by running an aliquot of the preparation on a gel, staining the DNA with a backbone stain, measuring the intensity of backbone staining in a region of the gel corresponding to DNA below a certain threshold, measuring the intensity of backbone staining in a region of the gel corresponding to DNA above a certain threshold, and comparing these to each other or to the total backbone staining intensity of the electrophoresed aliquot. The invention contemplates the use of the methods and/or reactors described herein to obtain such a population of nucleic acids.

The methods and/or reactors may also be used to maintain a population of nucleic acids without significant degradation or loss, optionally through one or more manipulations (including washes or changes in buffers), modifications (including labeling with a fluorophore or other detectable label) and/or reactions (including conjugation to another nucleic acid or to themselves). Thus, the invention provides a method comprising performing one or more buffer changes, washes, chemical and/or enzymatic reactions, or any other nucleic acid modification(s) or manipulation(s) to a population of nucleic acids without significant loss or degradation of the population of nucleic acids. The nucleic acid population may be small, in some instances. For example, the nucleic acid sample may be about 250-500 ng (optimal range) or 10-2000 ng (maximal range). In some embodiments, the nucleic acid sample may be less than or about 10 ng, 20 ng, 30 ng, 40 ng, 50 ng, 60 ng, 70 ng, 80 ng, 90 ng, 100 ng, 200 ng, 250 ng, 300 ng, 400 ng, 500 ng, 600 ng, 700 ng, 800 ng, 900 ng, 1000 ng, 1500 ng, or 2000 ng, or more.

It is to be understood that the total mass of biomaterial that may be added to the reactor initially may be far greater. As described elsewhere herein, the initial or starting sample may be a biomaterial such as, but not limited to, stool or food and as such will contain debris and/or other tissue or cellular material including proteins, polysaccharides, cell wall fragments and/or components, organelles, and the like. Thus the total mass of material introduced into a reactor may be orders of magnitude greater than the mass of the nucleic acids contained therein and being isolated by the method.

Thus, given that the starting mass of nucleic acids may be small (and/or the nucleic acids may be rare, including for example from a single cell or from a limited number of cells), it is important to minimize loss as much as possible. The reactors described herein are particularly suited for that purpose. Nucleic acid loss in the system may be less than 50%, less than 40%, less than 30%, less than 20%, less than 10%, less than 5%, less than 4%, less than 3%, less than 2%, or less than 1% of the starting nucleic acid amount. In some instances, nucleic acid loss in the system may be less than 0.09%, less than 0.08%, less than 0.07%, less than 0.06%, or less than 0.05% of the nucleic acid amount, including the starting nucleic acid amount. The degree of nucleic acid loss is related to the mass of nucleic acid, including the initial mass of nucleic acid. In some instances, larger losses are observed when the nucleic acid mass, including the initial or starting nucleic acid mass, is low (e.g., in the 10 ng range). Nucleic acid loss may be equal to or less than 10%, or equal to or less than 1%. Such a range of losses have been observed when the nucleic acid mass is in the 250-500 ng range. Loss in this regard may be measured by comparing the amount of nucleic acids introduced into the reactor and the amount of nucleic acids eluted from the reactor following the one or more manipulations, modifications and/or reactions.

The amount of nucleic acids may be measured using various methods known in the art. For example, the amount of nucleic acids may be measuring using spectroscopy. As is known in the art, nucleic acids such as DNA absorb light at wavelengths of about 260 nm. The amount of nucleic acids may also be measured through the size and intensity of nucleic acid bands in a gel, as described herein. Loss may be determined by comparing such bands. Still another method for measuring nucleic acid amount involves the use of an E-gel, as described in greater detail in Mollova et al. Anal. Biochem. 391: 135 (2009).

Degradation of nucleic acids may be detected through gel electrophoresis. In some instances, degradation may be detected and/or measured using the E-gel methodology described in Mollova et al. Anal. Biochem. 391: 135 (2009). Using this approach, large DNA (e.g., about equal to or greater than 100 kb) moves a single slow band and may be distinguished from shorter DNA.

In some embodiments, the initial nucleic acids may be about 50 kb to about 100 kb, or about 100 kb to about 500 kb, or about 500 kb to about 1000 kb, or about 100 kb to about 1000 kb. The invention is not limited in this regard, and the ability to manipulate nucleic acids, particularly rare nucleic acids and those that are available only in small starting quantities, will benefit from the reduced nucleic acid loss and degradation that is afforded by the methods and/or reactors of the invention. In some embodiments, a nucleic acid carrier may be added to the reactor in order to increase the overall nucleic acid mass, thereby reducing overall loss. In some instances, a suitable carrier is lambda phage DNA, or other such “neutral” DNA. By neutral DNA, it is intended that the DNA does not interfere with the manipulations, and any ultimate readout from such DNA may be readily excluded from the overall readout, thereby resulting in a dataset that is particular to the actual target nucleic acid of interest.

The manipulations, modifications and reactions described herein may include but are not limited to buffer changes, washes, restriction enzyme digestion, nick digestion, ligation, labeling with detectably labels, nucleic acid synthesis reaction such as a nick-fill reaction, hybridization to probes such as sequence-specific probes or PCR or other amplification probes, labeling with backbone stains, and the like. These and various other manipulations, modifications, and reactions are known in the art. In some embodiments, the number of manipulations, modifications and/or reactions is 2, 3, 4, 5, 6, 7, 8, 9, 10, 11, 12, 13, 14, 15, 16, 17, 18, 19, 20, or more.

In still other aspects, the invention provides populations of nucleic acids having a defined size range such as but not limited to about 100 kb to about 1000 kb (or any range therebetween, as described herein) in a composition that lacks any organic solvents. The methods of the invention yield nucleic acid populations suspended in solution (also referred to as “free-flowing”) and this renders the nucleic acids suitable for analyses such as linear analysis and the like.

In other aspects, the methods and/or reactors of the invention may be used to conjugate, such as covalently conjugate, the ends (or termini) of two nucleic acids to each other (e.g., conjugating a 3′ end of a first nucleic acid to 5′ of a second nucleic acid, or vice versa), wherein the first nucleic acid is at least 5-fold, 10-fold, 50-fold, 100-fold, 500-fold, 1000-fold (or more) longer than the second nucleic acid. Such covalent conjugation, in some instances, does not embrace crosslinking between or within one or more DNA.

In further aspects, the methods and/or reactors of the invention may be used to conjugate, such as covalently conjugate, two nucleic acids to each other (e.g., conjugating a 3′ end of a first nucleic acid to 5′ of a second nucleic acid, or vice versa), wherein the first nucleic acid and the second nucleic acid have a size in the range of about 10 kb to about 100 kb, or about 50 kb to about 100 kb, or about 100 kb to about 500 kb, or about 500 kb to about 1000 kb, or about 100 kb to about 1000 kb.

In some embodiments, the conjugations may be carried out in the presence of a ligase. Conjugation may involve blunt end conjugation or sticky-end conjugation.

In still other aspects, the invention provides methods for concentrating nucleic acids to densities on the order of nanogram/microliter (ng/μl). As an example, typically nucleic acids are eluted at concentrations of about 1 to about 10 ng/μl. Thus, the methods may be used to generate compositions having nucleic acid concentrations of equal to or less than about 10 ng/μl, 9 ng/μl, 8 ng/μl, 7 ng/μl, 6 ng/μl, 5 ng/μl, 4 ng/μl, 3 ng/μl, 2 ng/μl, or 1 ng/μl. It is to be understood that more dilute solutions may also be produced. In some embodiments, the nucleic acids are in the size range of about 10 kb to about 100 kb, or about 50 kb to about 100 kb, or about 100 kb to about 500 kb, or about 500 kb to about 1000 kb, or about 100 kb to about 1000 kb.

In some embodiments, the total amount of nucleic acids is in the range of about 250 ng to about 500 ng, or about 1000 ng to about 2000 ng, or about 250 ng to about 1000 ng, or about 250 ng to about 2000 ng, including various amounts therebetween as described elsewhere herein.

In some embodiments, the total volume of the compositions are in the range of about 50 μl to about 100 μl, although greater or less volumes are possible. For example, the volume may also be on the order of hundreds of microliters including in the 400-600 μl range. Thus, in some embodiments, the nucleic acids are provided in a volume that is less than or equal to about 40 μl, 50 μl, 60 μl, 70 μl, 80 μl, 90 μl, 100 μl, 200 μl, 300 μl, 400 μl, 500 μl, 600 μl, or more.

The methods and/or reactors may be used to isolate the resultant nucleic acid population (e.g., after all the modifications, manipulations and/or reactions are completed) to remove unreacted substrates or other entities that would other contaminate the nucleic acid composition or interfere with its intended use subsequent to its time in the reactor. The unreacted reagents may be probes, stains, enzymes, smaller nucleic acids, proteins such as antibodies or antibody fragments, and the like. The reactor can remove such entities. In this context, isolating the nucleic acids means physically separating the nucleic acids from other entities in the reactor. The degree of physical separation will depend on the degree that is required for the future use(s) of the nucleic acids. In some embodiments, it may be preferable to isolate the nucleic acids to a degree that facilitates their subsequent use and does not interfere with their future function.

Further, the invention contemplates manipulating, modifying, reacting, isolating and/or concentrating other moieties such as but not limited to proteins.

Any and all of the foregoing methods may be performed in short time frames including for example within 12 hours, within 8 hours, within 6 hours, within 5 hours, within 4 hours, within 3 hours, within 2 hours, within 1 hour, or less between when the nucleic acid is introduced into the reactor and when it is eluted from the reactor.

The methods and/or reactors provided herein, or any of those described in U.S. Pat. No. 8,361,716 B2, U.S. Publication No. 2010/0120101, U.S. Provisional Application No. 61/625,743 (filed Apr. 18, 2012), or Mollova et al. Anal Biochemistry, 391:135-143, 2009 may be used to isolate large intact fragments of nucleic acids (e.g., genomic and/or plasmid DNA) from samples (e.g., human biological samples or food samples) that may contain various microorganisms including, for example, bacteria, viruses and/or fungi. Nucleic acids isolated in accordance with the invention may be further analyzed by variety of techniques including but not limited to polymerase chain reaction (PCR), or other techniques where purified DNA is analyze; pulse field gel electrophoresis (PFGE), or other techniques where long intact purified DNA is analyzed; and Genome Sequence Scanning™, or other techniques where long intact purified sequence-specifically labeled DNA is analyzed.

Pulsed field gel electrophoresis is a technique used for the separation of large deoxyribonucleic acid (DNA) molecules by applying an electric field that periodically changes direction to a gel matrix. The procedure for this technique is similar to performing a standard gel electrophoresis except that instead of constantly running the voltage in one direction, the voltage is periodically switched among three directions: one that runs through the central axis of the gel and two that run at an angle of 60 degrees either side. The pulse times are equal for each direction resulting in a net forward migration of the DNA. For vary large bands (e.g., up to about 2 megabases), switching-interval ramps may be used to increases the pulse time for each direction over the course of a number of hours. For example, the pulse may be increased linearly from 10 seconds at 0 hours to 60 seconds at 18 hours. This procedure takes longer than normal gel electrophoresis due to the size of the fragments being resolved and the fact that the DNA does not move in a straight line through the gel.

Genome Sequence Scanning™ (GSS™), also referred to as Direct Linear Analysis (DLA), was developed by PathoGenetix, Inc. for the analysis of long double-stranded DNA (see, e.g., Mollova et al. 2009; White et al. Clin. Chemistry, 55:2121-2119, 2009; and Protozanova et al. Anal Biochemistry, 402:83-90, 2010).

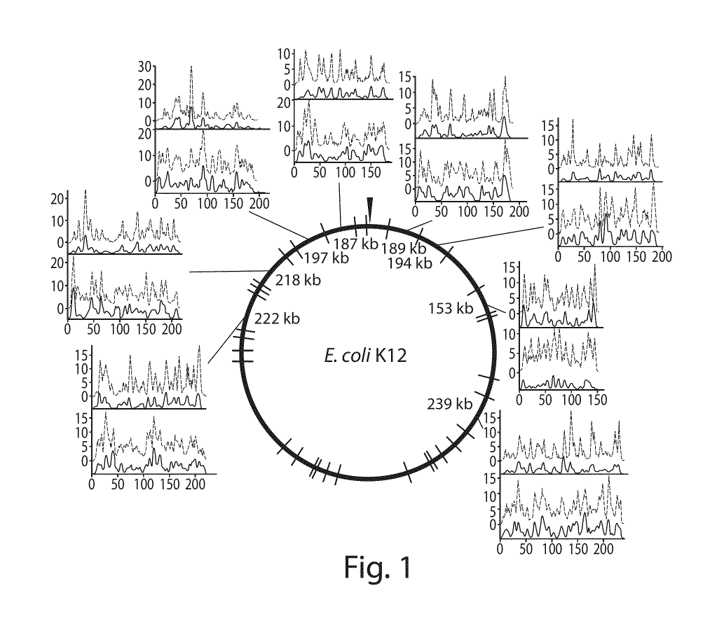

GSS™ analysis includes the isolation of genomic DNA followed by its digestion with rare-cutting endonucleases to obtain long DNA fragments (e.g., ˜80-350 kb). The DNA is then probed with two types of fluorescent marker probes (e.g., ˜6-8 bases): one that binds to DNA non-specifically and defines the presence of a DNA fragment; and another that binds to DNA in a sequence-specific manner and forms a trace defined by the underlying genomic DNA sequence (FIG. 1). The latter can be compared to a database for identifying the microorganism from which the DNA fragment originated.

The endonuclease used in accordance with the invention should produce several DNA fragments within this an approximately 80 kb to 350 kb range per genome. Every fragment carries multiple sequence-specific tags producing microorganism-specific maps. These maps can be obtained in two colors by targeting different sequences on DNA with two tags labeled with spectrally resolved fluorescent dyes. This increases the information content on single molecules and increases confidence of microorganism identification.

Database used for microorganism identification may be calculated from genome sequence or measured using cultured isolate. Only microorganisms that are present in the database can be identified. Because there is a sufficient overlap between genomes of related microorganisms, unknown strains can also be identified as new, but related to certain microorganism present in a database.

### Reactors/Devices

Reactors and devices that may be used in accordance with the invention include those described herein as well as any of those described in U.S. Pat. No. 8,361,716 B2, U.S. Publication No. 2010/0120101, U.S. Provisional Application No. 61/625,743 (filed Apr. 18, 2012), and Mollova et al. Anal Biochemistry, 391:135-143, 2009, each of which is incorporated by reference herein in its entirety. The terms “reactor” and “device” may be used interchangeably herein.

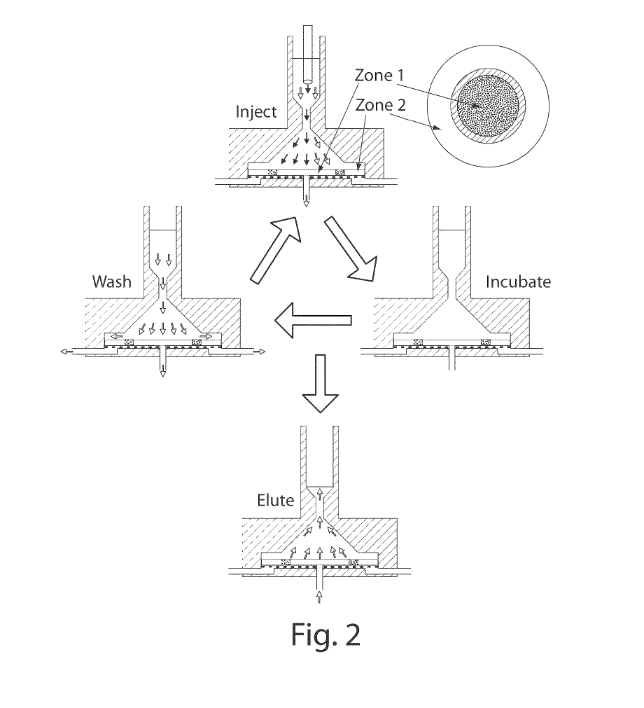

A description of several examples of reactors and devices that may be used in accordance with the invention is provided below. A reactor may contain a porous substrate onto which the sample is deposited (zone 1) and an inverted cone body (FIG. 2). The circumference of the base of the cone may have multiple pedestals which create channels for fluidic connections to zone 2. The reactor may be filled with liquid (e.g., ethanol, various buffer solutions, various lytic solutions, and/or various digest solutions). The liquid can be replaced with a different liquid by applying vacuum (i) across the porous substrate in zone 1 thereby creating liquid flow normal to the substrate (through the sample) and (ii) across the substrate in zone 2 thereby creating liquid flow tangential to the membrane (above the sample). Different solutions and reagents may be introduced into the reactor from the reactor top. Large objects like cells and genomic DNA are immobilized on the porous substrate by applying small flow through the substrate in zone 1. Optimized flows in zone 1 and zone 2 allow for fast exchange of solutions within the reactor, fast and efficient and uniform delivery of reagent to substrates, and fast and efficient removal of debris and reaction by-products. The latter is accomplished by two means: small molecules are removed through the porous substrate (e.g., oligopeptides), and larger molecules are removed by a flow tangential to the substrate (e.g., polypeptides larger than 100 kD). Larger objects remain on the substrate (e.g., cells, genomic DNA). Examples of porous substrates for use in accordance with the invention include membranes such as, for example, ultrafiltration (UF) membranes (e.g., polyethersulfone, polyacrylonitrile, polyvinylidene UF membranes), including those with 100 kD MWCO.

The reactor may also be equipped with temperature control which operates from room temperature to about 75° C. The reactor is further equipped with the ability to reverse flow through membrane in zone 1 thereby permitting elution of prepared DNA from the membrane through the top of the cone for further analysis.

### EXAMPLES

### Automated Sample Preparation Method

Generally, the sample preparation methods provided herein comprises at least one of the steps listed in Table I. The following parameters may vary, depending on the sample type: reagents and chemistries, time of each step (e.g., injection, incubation, wash), temperature at which each step is performed, and order of the steps.

TABLE I

| Step | Process |
|---|---|
| Sample injection | deposit microorganism(s) on membrane |
| Sample lysis | wash with lytic buffer for 2 to 10 minutes |
| | injection of lytic reagents |
| | incubation in presence of lytic reagents |
| | for t (min) at T (° C.) |
| DNA digestion | wash with restriction enzyme buffer for |
| | 2 to 10 min |
| | injection of restriction enzyme |
| | incubation in presence of restriction |
| | enzyme for t (min) at T (° C.) |
| DNA tagging | wash with low salt buffer for 2 to 10 min |
| e.g., for GSS ™) | injection of bisPNAs |
| | incubation in presence of bisPNAs for t |
| | (min) at T (° C.) |
| | wash with high salt buffer for 2 to 10 min |
| | incubation in presence of high salt buffer |
| | for t (min) at T (° C.) temperature |
| | wash with low salt buffer for 2 to 10 min |
| DNA elution | wash with buffer compatible with further |
| | analysis for 2 to 10 minutes (optional) |
| | reverse flow through the membrane and |
| | collect eluted DNA on top of cone |

### Lytic Step(s)

The number of and order of the lytic step(s) may be adjusted to target one or more microorganisms or mixtures of microorganisms. The number of and order of the lytic step(s) may also be adjusted based on the type of organism. For example, particular lytic reagents and chemistries may be based on the structure and chemistry of the particular microorganism. Additionally, some of the lytic reagents can act together in a single reaction (e.g., lysozyme and achromopeptidase), while others may require a separate reaction (e.g., proteinase K).

### Example 1

### Single Lysis Method: Isolation of High Quality DNA from E. coli K12

TABLE II

| | | | Time of | Temperature |
|---|---|---|---|---|
| | | Can contain | incubation, | of incubation, |
| Step | Reagent | chemistry | min | ° C. |
| Sample | 50-500 μg | 0.1-1% w/v SDS | 5-30 or | 37-75 |
| lysis | Proteinase K | 1-50 mM EDTA | 10-30 | |
| | | 0-2% v/v BME | | |
| | | 0-7M Urea | | |
| | | 10-50 mM Tris, | | |
| | | pH 6-8 | | |
| Sample | 50-500 μg | 0.1-1% w/v SDS | 5-30 or | 37-75 |
| lysis | Proteinase K | 1-50 mM EDTA | 10-30 | |
| | | 0-2% v/v BME | | |
| | | 0-7M Urea | | |
| | | 10-50 mM Tris, | | |
| | | pH 6-8 | | |

### Example 2

### Single Lysis Method: Isolation of High Quality DNA from Staphylococcus aureus Spp

TABLE III

| | | Can contain | | |
|---|---|---|---|---|
| | Reagent | chemistry | Time of | Temperature |
| | (other details | (other details | incu- | of incu- |
| | specified in | specified | bation, | bation, |
| Step | Example 1) | in Example 1) | min | ° C. |
| Sample | 5-50 μg | 0.1-1% v/v | 5-30 or | 37-55 |
| lysis | Lysostaphin | Tween20 | 10-30 | |
| | | 0.1-1% v/v | | |
| | | Triton-X-100 | | |
| | | 10-50 mM EDTA | | |
| | | 10-50 mM Tris, | | |
| | | pH 6-8 | | |
| Sample | Proteinase K | SDS, EDTA, BME, | 5-30 or | 37-75 |
| lysis | | Urea, Tris | 10-30 | |
| Sample | Proteinase K | SDS, EDTA, BME, | 5-30 or | 37-75 |
| lysis | | Urea, Tris | 10-30 | |

### Example 3

### Double Lysis Method: Isolation of High Quality DNA from E. coli and Bacillus Spp

TABLE IV

| | | Can contain | | |
|---|---|---|---|---|
| | Reagent | chemistry | Time of | Temperature |
| | (other details | (other details | incu- | of incu- |
| | specified in | specified in | bation, | bation, |
| Step | Example 1-2) | Example 1-2) | min | ° C. |
| Sample | Proteinase K | SDS, EDTA, | 5-30 or | 37-75 |
| lysis | | BME, Urea, | 10-30 | |
| | | Tris | | |
| Sample | Lysozyme, | Tween20, | 5-30 or | 37-55 |
| lysis | 50-500 units | Triton-X-100, | 10-30 | |
| | Achromopep- | EDTA, Tris | | |
| | tidase, 50- | | | |
| | 500 units | | | |
| | Mutanolysin | | | |
| Sample | Proteinase K | SDS, EDTA, | 5-30 or | 37-75 |
| lysis | | BME, Urea, | 10-30 | |
| | | Tris | | |
| Sample | Proteinase K | SDS, EDTA, | 5-30 or | 37-75 |
| lysis | | BME, Urea, | 10-30 | |
| | | Tris | | |
| Sample | Lysozyme | Tween20, | 5-30 or | 37-55 |
| lysis | 50-500 μg | Triton-X-100, | 10-30 | |
| | Labiase | EDTA, Tris | | |
| Sample | Proteinase K | SDS, EDTA, | 5-30 or | 37-75 |
| lysis | | BME, Urea, | 10-30 | |
| | | Tris | | |
| Sample | Proteinase K | SDS, EDTA, | 5-30 or | 37-75 |
| lysis | | BME, Urea, | 10-30 | |
| | | Tris | | |

### Example 4

### Triple Lysis Method: Isolation of High Quality DNA from E. coli and Bacillus Spp* and Yeast

(NOTE: *vegetative cells or de-coated spores; a spore de-coating method for use in accordance with the invention is described in U.S. Pat. No. 7,888,011 B2, incorporated herein by reference in its entirety.)

TABLE V

| | | Can contain | | |
|---|---|---|---|---|
| | Reagent | chemistry | Time of | Temperature |
| | (other details | (other details | incu- | of incu- |
| | specified in | specified in | bation, | bation, |
| Step | Example 1-3) | Example 1-3) | min | ° C. |
| Sample | Proteinase K | SDS, EDTA, BME, | 5-30 or | 37-75 |
| lysis | | Urea, Tris | 10-30 | |
| Sample | Lysozyme, | Tween20, Triton- | 5-30 or | 37-55 |
| lysis | Achromopep- | X-100, EDTA, | 10-30 | |
| | tidase, | Tris | | |
| | Mutanolysin | | | |
| Sample | Proteinase K | SDS, EDTA, BME, | 5-30 or | 37-75 |
| lysis | | Urea, Tris | 10-30 | |
| Sample | Lysozyme, | Tween20, Triton- | 5-30 or | 37-55 |
| lysis | Labiase | X-100, EDTA, | 10-30 | |
| | | Tris | | |
| Sample | Proteinase K | SDS, EDTA, BME, | 5-30 or | 37-75 |
| lysis | | Urea, Tris | 10-30 | |
| Sample | Lysozyme, | Tween20, Triton- | 5-30 or | 37-55 |
| lysis | 50-500 units | X-100, EDTA, | 10-30 | |
| | Lyticase | Tris | | |
| Sample | Proteinase K | SDS, EDTA, BME, | 5-30 or | 37-75 |
| lysis | | Urea, Tris | 10-30 | |
| Sample | Proteinase K | SDS, EDTA, BME, | 5-30 or | 37-75 |
| lysis | | Urea, Tris | 10-30 | |

### DNA Digestion Step(s)

DNA digestion step(s) may be adjusted to target one or more microorganisms or mixtures of microorganisms. DNA digestion step(s) may include one digestion step in instances where use of one restriction enzyme produces DNA fragments within the length range required for further analysis. The DNA digestion protocol may also include one digestion step in instances where two restriction enzymes, acting together in the same buffer conditions, produce a double digest of DNA with fragments within the length range required for further analysis.

When multiple restriction digests are required, for example, to sub-type closely related E. coli strains (see e.g., Ribot, E M et al. Foodborne Pathogens and Disease. 3: 59-67, 2006; Gupta, A et al. Emerg Infect Dis. 10: 1856-1858, 2004) or to target multiple microorganisms with a wide range of GC content in the same assay, a DNA digestion step, using multiple restriction enzymes, may be performed more than once (e.g., multiple DNA digestion steps).

Alternatively, a DNA digestion step may be conducted in one reaction as outlined below in Example 7. In Example 7, restriction enzyme 1 (RE1) is injected in a sheath buffer stream so that it covers only a portion of the purified DNA on the membrane of the reactor. This injection is accomplished by positioning the probe, which delivers restriction enzyme to the reactor, off the center of the reactor inlet (FIG. 6A). Only DNA that comes in contact with the restriction enzyme is digested (FIG. 6B). Digested DNA is selectively eluted from the membrane by reversing the flow through the membrane. Undigested genomic DNA is very long and entangled in the close proximity of the membrane, so it remains on the membrane during gentle elution at 20-500 μl/cm² (FIG. 6B). Restriction enzyme 2 (RE2) is then injected so that it comes in contact with remaining DNA on membrane. DNA is eluted following digestion. This DNA digestion step yields two restriction digests of the same DNA which then may be analyzed separately or combined for further analysis (FIG. 6C).

### Example 5

### DNA Digestion with One Restriction Enzyme, or Mixture of Restriction Enzymes to Produce Double Digested DNA

(NOTE: buffer conditions are optimal for activity of all restriction enzymes, if more than one is used.)

TABLE VI

| | | | | Temper- |
|---|---|---|---|---|
| | | | Time of | ature of |
| | | | incu- | incu- |
| | | Can contain | bation, | bation, |
| Step | Reagent | chemistry | min | ° C. |
| DNA | 1-100 units | 0-10 mM | 5-30 or | 25-37 |
| digestion | Restriction | Magnesium salt, | 10-30 | |
| | enzyme(s), | 0-200 mM Sodium | | |
| | 0-10 ng | salt, 0-200 mM | | |
| | RNase | Potassium salt, | | |
| | | 10-50 mM Tris, | | |
| | | pH 6-8 | | |

### Example 6

### Protocol to Obtain Double Digest of DNA with Restriction Enzymes Requiring Different Buffer Conditions for Optimal Activity can Contain Following Steps

TABLE VII

| | | Can contain | Time of | Temperature |
|---|---|---|---|---|
| | Reagent | chemistry | incu- | of incu- |
| | (details same | (details same | bation, | bation, |
| Step | as Example 5) | as Example 5) | min | ° C. |
| DNA | Restriction | Magnesium, | 5-30 or | 25-37 |
| digestion | enzyme 1, | sodium, | 10-30 | |
| | RNase | potassium, | | |
| | | Tris | | |
| DNA | Restriction | Magnesium, | 5-30 or | 25-37 |
| digestion | enzyme 2, | sodium, | 10-30 | |
| | RNase | potassium, | | |
| | | Tris | | |

### Example 7

### Protocol for Digestion of DNA with Two or More Restriction Enzymes in the Same Reactor

TABLE VIII

| | | Can contain | Time of | Temperature |
|---|---|---|---|---|
| | Reagent | chemistry | incu- | of incu- |
| | (details same | (details same | bation, | bation, |
| Step | as Example 5) | as Example 5) | min | ° C. |
| DNA | Restriction | Magnesium, | 5-30 or | RT-37 |
| digestion* | enzyme 1, | sodium, | 10-30 | |
| | RNase | potassium, | | |
| | | Tris | | |
| DNA | | | | |
| elution | | | | |
| DNA | Restriction | Magnesium, | 5-30 or | RT-37 |
| digestion** | enzyme 2, | sodium, | 10-30 | |
| | RNase | potassium, | | |
| | | Tris | | |
| DNA | | | | |
| elution | | | | |

*Restriction enzyme 1 is injected in sheath flow to cover only portion, approximately half, of the purified DNA on the membrane. Only DNA that is in contact with restriction enzyme is digested. The rest of purified DNA remains intact and will not be eluted due to physical entanglement.
**Restriction enzyme 2 is injected in sheath flow to cover and act on the remaining DNA.

### DNA Tagging Step(s)

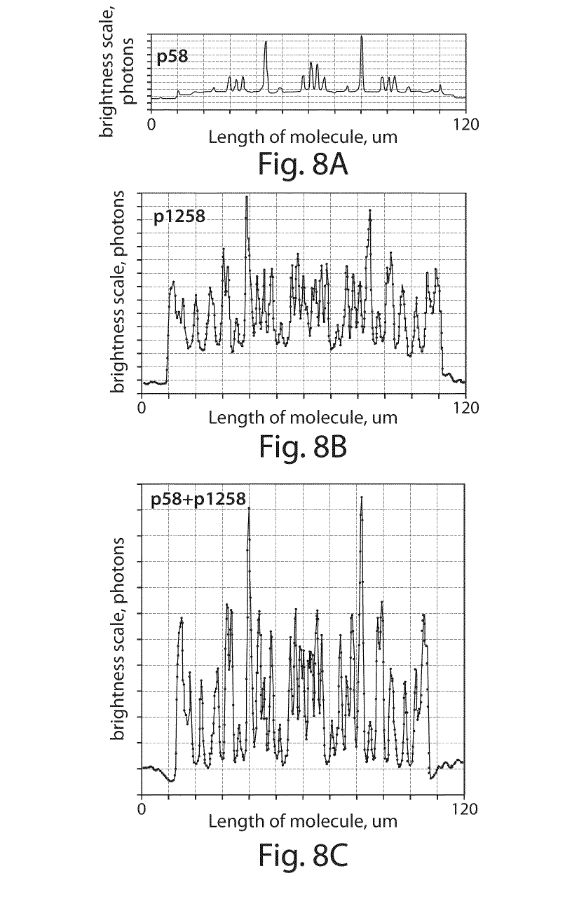

A sequence-specific tagging step used in GSS for microorganism identification may be adjusted to target one or more microorganisms or mixtures of microorganisms. The choice of the tag (bisPNA) depends on GC content of microorganisms or their mixtures. Generally, bisPNA complementary to sites on DNA with more guanines may be used to target higher GC-content genomic DNA. BisPNA complementary to sites on DNA with less guanines may be used to target lower GC-content genomic DNA (AT-rich). FIG. 8A shows fluorescent trace of a fragment from a GC-rich genome obtained with a tag complementary to the 5′-GAAGAAAA sequence on DNA. This tag has only one binding site on a 201 kb fragment, which is inadequate for fragment identification by GSS. Alternatively, a tag complementary to the 5′-GAAGAAGG sequence on DNA has 14 binding sites on the same fragment and therefore a much more rich profile for GSS analysis (FIG. 8B).

To increase information carried by each molecule, GSS analysis can employ two spectrally resolved kinds of tags in the same reaction (Protozanova et al. 2010), for example, tags labeled with ATTO550 and ATTO647N or other spectrally resolved pair of fluorophores. If an analysis includes microorganisms with genomes with a narrow range of GC-contents, both tags may target either GC-rich, neutral or AT-rich genomes in one reaction. If the analysis includes both, GC-rich and AT-rich genomes simultaneously, one tag may target GC-rich genomes and the other tag may target AT-rich genomes in one reaction.

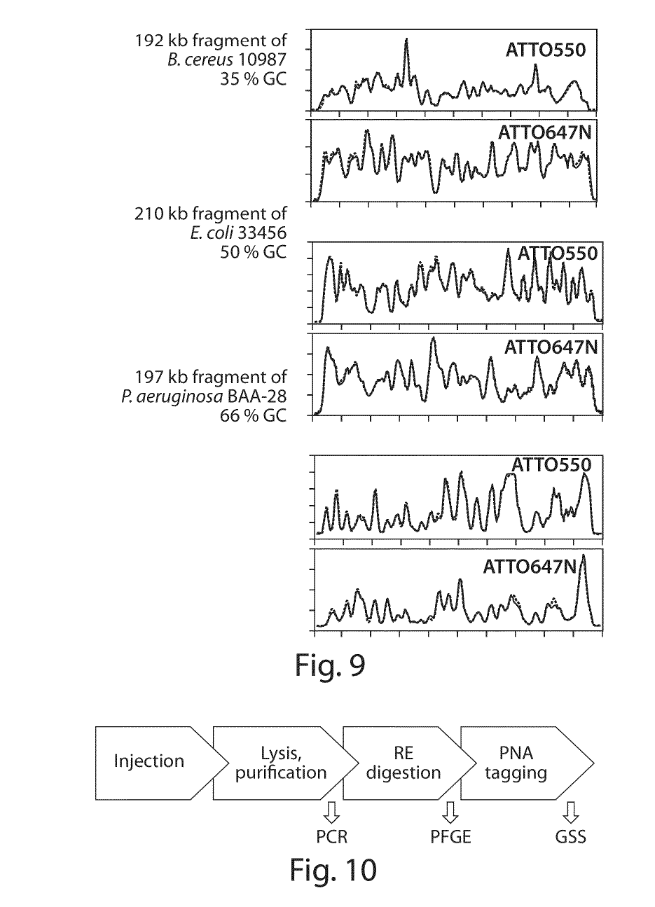

Alternatively, if the analysis includes both, GC-rich and AT-rich genomes simultaneously, a mixture of two tags, which are not spectrally resolved, may be used in the same reaction (FIG. 8C). This mixture may contain one or more tags that target GC-rich genomes and one or more tags that target AT-rich genomes. To increase information carried by each molecule, the latter mixture of tags may also be appended with one or more tags that target GC-rich genomes and one or more tags that target AT-rich genomes, both spectrally resolved from the first mixture. FIG. 9 shows GSS traces for GC-rich, neutral and AT-rich genomes obtained with the same “universal” mixture of tags.

### Example 8

### Protocol for DNA Tagging with bisPNA, or a Mixture of bisPNAs

TABLE IX

| | | | | Temperature |
|---|---|---|---|---|
| | | | Time, | of incubation, |
| Step | Reagent | Can contain chemistry | min | ° C. |
| DNA | 0.1-2 μM | 1^{st} incubation: | 5-60 or | 37-75 |
| tagging | PNAs | 10-50 mM Tris pH 6-8 | 10-60 | |
| | | 1-5 mM EDTA | | |
| | | 2^{nd} incubation: | 5-60 or | 37-75 |
| | | 10-50 mM Tris pH 6-8 | 10-60 | |
| | | 1-5 mM EDTA | | |
| | | 50-500 mM Sodium salt | | |

The methods of the invention may be adjusted as needed to produce DNA ready for further analysis by various techniques (FIG. 10).

DNA can be eluted from the reactor following sample lysis, DNA digestion and DNA tagging for analysis by GSS or other techniques where long intact purified sequence-specifically labeled DNA is analyzed. Genomic DNA digested with restriction enzyme is readily eluted from membrane by gentle reverse flow of 20-500 μl/cm². Elution protocol entails washing DNA with solution compatible with further analysis, reversing the flow through the membrane and collecting DNA suspended in solution at the top of reactor.

DNA can be eluted from the reactor following sample lysis and DNA digestion for analysis by PFGE or other techniques where long intact purified DNA is analyzed. The elution protocol may be similar to the one described above.

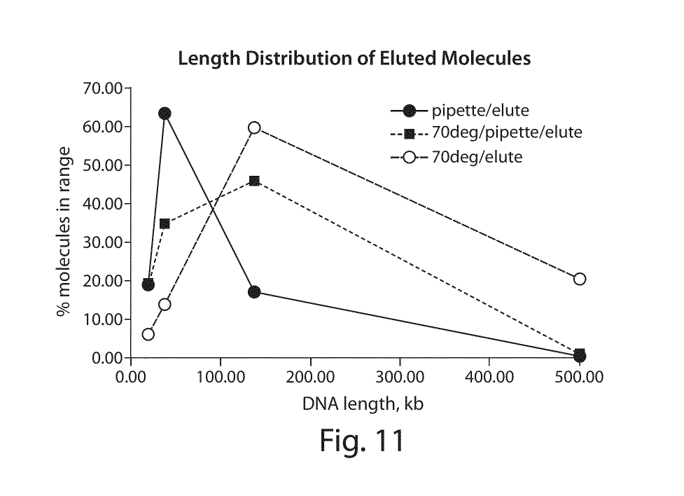

DNA can be eluted from the reactor following sample lysis for analysis by PCR or other techniques that utilize purified DNA. Purified genomic DNA is entangled close to the membrane and is not eluted efficiently by reverse flow. For efficient elution, the DNA may be damaged by incubating the DNA at elevated temperature (65-75° C.) for 5-60 or 10-60 minutes under low salt conditions prior to elution. Alternatively, the DNA may be damaged by shear introduced by repeated aspirating and dispensing the solution close to membrane prior to elution. The DNA also may be damaged by a combination of the above prior to gentle elution at 20-500 μl/cm². FIG. 11 shows DNA length distribution following each of the three treatments. In all cases DNA fragments of at least 20 kb, at least 50 kb, at least 100 kb are recovered, and at least 200 kb were recovered. In some instances, DNA fragments up to 500 kb were recovered.

### Example 9

### Sample Preparation Method of the Invention

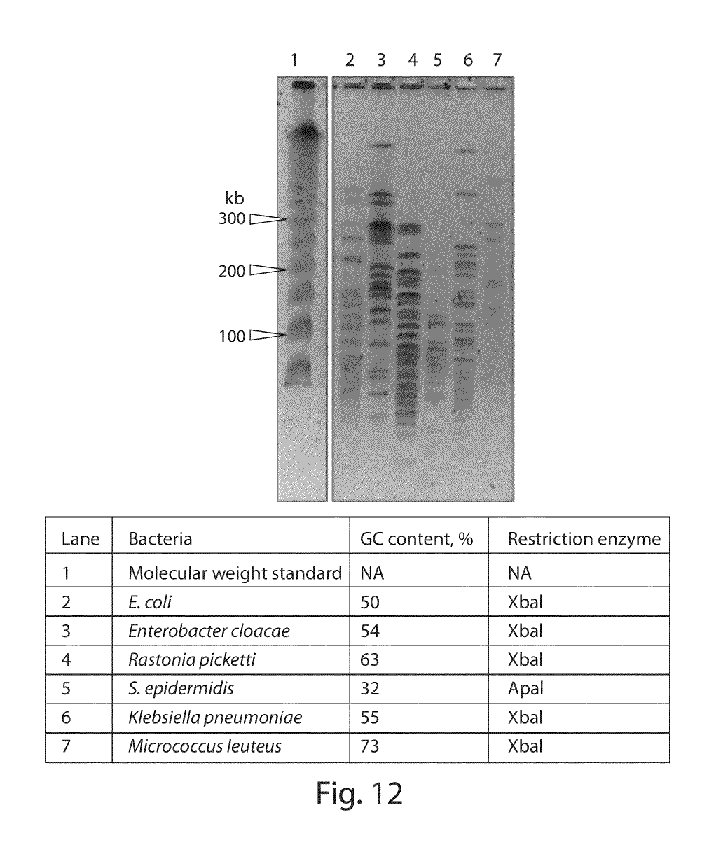

A pulse field gel electrophoresis (PFGE) band pattern shows high molecular weight genomic DNA extracted from various Gram positive and Gram negative bacteria, with GC-rich and AT-rich genomes (FIG. 12). The double lysis method described in Example 3 was used for cell lysis.

### Example 10

### Sample Preparation Method of the Invention

Samples of ground beef with and without spiking with 1000 cfu of Salmonella was incubated in buffer/broth overnight with shaking. The flask was then removed from the shaker and permitted to stand for 30 min A pipette was then used to remove 2 ml of the overnight culture, avoiding large particulates, and the 2 ml of culture was dispensed in a 2 ml vial.

A bench top centrifuge was used to spin 0.5-2.0 ml of the sample suspension at 15-30 relative centrifugal force (rcf) for 5-30 minutes at room temperature (˜20° C. to 25° C.). The supernatant was transferred to a fresh 2 ml vial, and the vial with the pellet was discarded. A bench-top centrifuge was then used to spin the vial with the supernatant at 13000 rcf for 2-15 minutes at room temperature. The supernatant was discarded.

The pellet was resuspended in 2 ml of buffer/broth by pipetting. The cells were spun down by centrifugation at 13000 rcf for 2-3 minutes at room temperature. The supernatant was discarded. The wash procedure was repeated 1-3 times.

The pellet was resuspended in buffer/broth to a concentration corresponding to 0.1 to 2 OD.

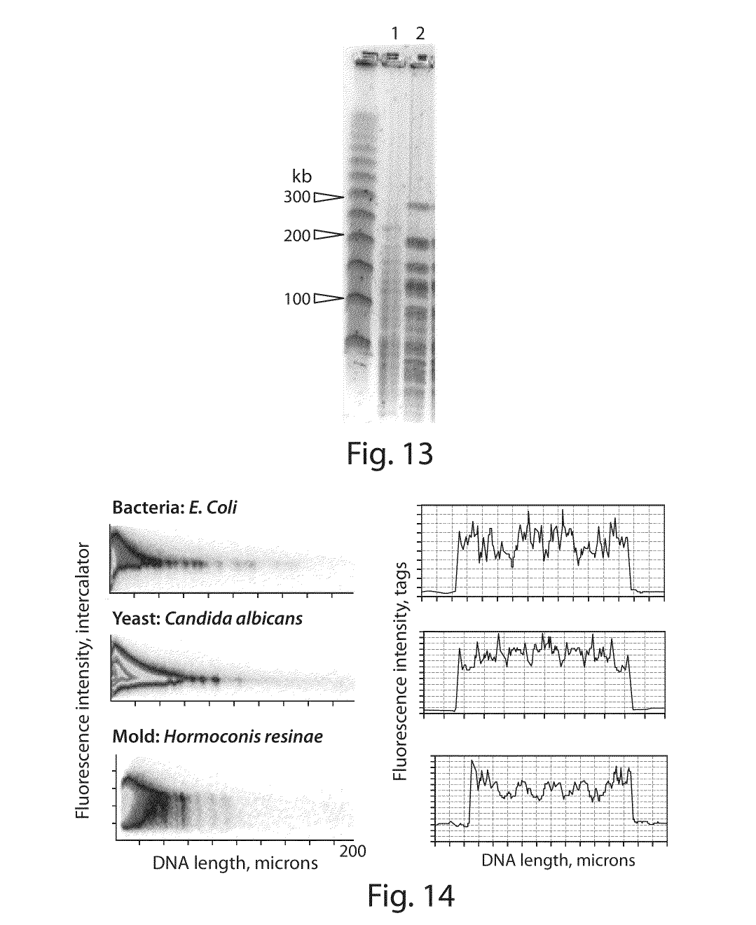

A PFGE band pattern showed high molecular weight genomic DNA extracted from the ground beef sample (FIG. 13).

### Example 11

### Sample Preparation Method of the Invention

GSS analysis of bacteria, yeast and mold prepared using a triple lysis step (see Example 4) is shown in FIG. 14. Hyphae was separated from the mold culture using the following pre-treatment method of the invention:

0.5-2 ml of mold culture having an optical density (OD) of 10 was resuspended in 4 ml of buffer/broth containing 10% HistoDenz™. The entire volume was layered onto a step gradient in a 15 ml Falcon tube that included 0.5-2 ml of 30% HistoDenz™ in buffer/broth, 0.5-2 ml of 40% HistoDenz™ in buffer/broth, and 0.5-2 ml of 60% HistoDenz™ in buffer or broth (i. e., 10-30-40-60 gradient). Following centrifugation, a band at the 30-40% interface was recovered and processed by the triple lysis protocol.

### Other Embodiments

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Each of the references recited herein is incorporated herein by reference in its entirety.

1. A method of isolating nucleic acid using a chamber having a porous substrate, the method comprising:
(a) flowing a sample comprising cells through a fluid port and onto a porous substrate in the chamber;
(b) flowing lytic buffer solution through a fluid port and through the porous substrate in the chamber; flowing a fluid containing lytic reagents through the fluid port and onto the porous substrate in the chamber, and incubating the porous substrate at a first temperature for a first period of time;
(c) flowing an endonuclease buffer solution through the fluid port and through the porous substrate in the chamber; flowing a fluid containing digest reagents through the fluid port and onto the porous substrate in the chamber, and incubating the porous substrate at a second temperature for a second period of time; and
(d) reversing flow through the fluid port to move any nucleic acids positioned on the porous substrate out of the chamber in central streamlines that exit the chamber through the fluid port, thereby isolating the nucleic acids,
wherein the cells are optionally microorganisms.

2. The method of claim 1, wherein the lytic reagents comprise a lytic enzyme.

3. The method of claim 2, wherein the lytic enzyme is proteinase K, lysostaphin, lysozyme, achromopeptidase, mutanolysin, or any combination of two or more of the foregoing.

4. The method of claim 1, wherein the lytic reagents further comprise a buffer, a denaturing agent, a detergent, a chelating agent, a reducing agent, or any combination of two or more of the foregoing.

5. The method of claim 1, wherein the first temperature of (b) is about 37° C. to about 75° C.

6. The method of claim 1, wherein the first period of time of (b) is about 10 minutes to about 30 minutes.

7. The method of claim 1, wherein (b) is performed multiple times.

8. The method of claim 7, wherein multiple different lytic enzymes are used and multiple different lytic buffer solutions are used.

9. The method of claim 7, wherein (b) comprises:
flowing a fluid containing lytic reagents through the fluid port and onto the porous substrate in the chamber, wherein the lytic reagents comprise a first lytic enzyme and a first lytic buffer solution, and incubating the porous substrate; and
flowing a fluid containing lytic reagents through the fluid port and onto the porous substrate in the chamber, wherein the lytic reagents comprise a second lytic enzyme different from the first lytic enzyme and a second lytic buffer solution different from the first lytic buffer solution, and incubating the porous substrate.

10. The method of claim 1, wherein the digest reagents comprise an endonuclease.

11. The method of claim 10, wherein the endonuclease is PmeI, XbaI, ApaI, or any combination of two or more of the foregoing.

12. The method of claim 10, wherein the digest reagents further comprise magnesium, sodium, potassium, salt, tris(hydroxymethyl)aminomethane or any combination of two or more of the foregoing.

13. The method of claim 1, wherein the second temperature of (c) is about 20° C. to about 37° C.

14. The method of claim 1, wherein the second period of time of (c) is about 10 minutes to about 30 minutes.

15. The method of claim 1, wherein (c) is performed multiple times, in consecutive order.

16. The method of claim 15, wherein multiple different endonucleases are used and multiple different endonuclease buffer solutions are used.

17. The method of claim 15, wherein (c) comprises:
flowing a fluid containing digest reagents through the fluid port and onto the porous substrate in the chamber, wherein the digest reagents comprise a first endonuclease and a first endonuclease buffer solution, and incubating the porous substrate; and
flowing a fluid containing digest reagents through the fluid port and onto the porous substrate in the chamber, wherein the digest reagents comprise a second endonuclease different from the first endonuclease and a second endonuclease buffer solution different from the first endonuclease buffer solution, and incubating the porous substrate.

18. The method of claim 1, further comprising flowing low salt wash buffer through the fluid port and through the porous substrate in the chamber, flowing a fluid containing nucleic acid probe through the fluid port and onto the porous substrate in the chamber, incubating the porous substrate at a third temperature for a third period of time, flowing high salt wash buffer through the fluid port and on the porous substrate in the chamber, incubating the porous substrate at a fourth temperature for a fourth period of time, and flowing low salt wash buffer through the fluid port and through the porous substrate in the chamber.

19. The method of claim 1, wherein the sample is pre-treated to remove matrix.

20. The method of claim 19, wherein the matrix is removed by sedimentation, selective sedimentation, density gradient centrifugation or filtration.

21. The method of claim 1, wherein the sample is a biological sample.

22-23. (canceled)

24. The method of claim 1, wherein the nucleic acids have a length of at least 50 kilobases, at least 100 kilobases, at least 150 kilobases, at least 250 kilobases, at least 500 kilobases, at least 750 kilobases, at least 1 megabase, or at least 5 megabases.

25. (canceled)

26. The method of claim 1, wherein the nucleic acids are isolated in 6 hours or less, 5 hours or less, 4 hours or less, or 3 hours or less.

27. The method of claim 1, wherein the sample comprises microorganisms selected from the group consisting of bacteria, fungi, viruses or a combination of any two or more of the foregoing.

28. (canceled)

29. The method of claim 1, wherein the sample is not cultured prior to flowing the sample through the fluid port, or wherein the sample is a cultured isolate, or wherein the sample is a mixture thereof.

30. The method of claim 1, wherein the porous substrate is a membrane.

31. The method of claim 30, wherein the membrane is an ultrafiltration membrane.

32. A method of isolating nucleic acid using a chamber having a porous substrate, the method comprising:
(a) flowing a sample comprising cells through a fluid port and onto a porous substrate in the chamber;
(b) flowing lytic buffer solution through a fluid port and through the porous substrate in the chamber; flowing a fluid containing lytic reagents through the fluid port and onto the porous substrate in the chamber, and incubating the porous substrate at a first temperature for a first period of time;
(c) flowing a first endonuclease buffer solution through the fluid port; flowing a fluid containing digest reagents through the fluid port off-center and onto the first half of the porous substrate in the chamber, and incubating the porous substrate at a second temperature for a second period of time;
(d) reversing flow through the fluid port to move any nucleic acids positioned on the first half of porous substrate out of the chamber through the fluid port, thereby isolating digested nucleic acids on the first half of the porous substrate;
(e) flowing a second endonuclease buffer solution through the fluid port; flowing a fluid containing digest reagents through the fluid port off-center and onto the second half of the porous substrate in the chamber, and incubating the porous substrate at a third temperature for a third period of time; and
(f) reversing flow through the fluid port to move any nucleic acids positioned on the second half of porous substrate and therefore digested with second digest reagent out of the chamber through the fluid port, thereby isolating the nucleic acids from the second half of the porous substrate,
wherein optionally the cells are microorganisms.

33-71. (canceled)

72. A method of isolating nucleic acid using a chamber having a porous substrate, the method comprising:
(a) flowing a cell population comprising through a fluid port and onto a porous substrate in the chamber;
(b) flowing lytic buffer solution through a fluid port and through the porous substrate in the chamber; flowing a fluid containing lytic reagents through the fluid port and onto the porous substrate in the chamber, and incubating the porous substrate at a set temperature for a set period of time to release nucleic acids from the cell population;
(c) (i) incubating the porous substrate at a temperature of about 65° C. to about 75° C. for a time sufficient to permit melting of AT-rich regions of the nucleic acid; or
(ii) aspirating solution from the chamber and depositing the solution onto the porous substrate, and optionally repeating the aspirating and depositing multiple times thereby shearing the nucleic acid; and
(d) reversing flow through the fluid port to move any nucleic acids positioned on the porous substrate out of the chamber in central streamlines that exit the chamber through the fluid port, thereby isolating the nucleic acids.

73. (canceled)

74. The method of claim 72, wherein the portion that contains nucleic acid fragments having lengths in the range of about 100 kb to about 1000 kb represents at least about 50%, at least about 60%, at least about 70%, at least about 80%, at least about 90%, or at least about 100% of the population.

75-79. (canceled)
